(19) United States
(12) Patent Application Publication
(10) Pub. No.: US 2019/0228848 A1
(43) Pub. Date: Jul. 25, 2019

(54) SYSTEMS, DEVICES, AND METHODS FOR GENERATING A MEDICAL NOTE INTERFACE

(71) Applicant: OutcomeMD, Inc., Portland, OR (US)

(72) Inventor: Justin Saliman, Portland, OR (US)

(21) Appl. No.: 16/258,363

(22) Filed: Jan. 25, 2019

Related U.S. Application Data
(60) Provisional application No. 62/622,020, filed on Jan. 25, 2018.

(51) Int. Cl. G16H 10/60; G16H 10/20; G16H 50/30

(52) U.S. Cl. CPC G16H 10/60; G16H 50/20; G16H 50/30; G16H 10/20

(57) ABSTRACT

Systems, methods, and devices for generating one or more medical note interfaces may receive responses to a plurality of questions included in a medical questionnaire and prepare a medical note graphic user interface that includes the received responses. In some instances, each of the received responses may be concurrently displayed with a correlated question of the plurality of questions. medical note interface may be provided to a user. Then, an indication that the user has reviewed the medical note interface may be received and the medical note interface may be locked. In some embodiments, a wellness score and/or improvement score may be determined for the patient based on the received responses. These wellness and/or improvement scores may be included in a medical note interface provided to the user.

### RELATED APPLICATION

The present application is a NON-PROVISIONAL of, and claims priority to, U.S. Provisional Patent Application No. 62/622,020, filed on 25 Jan. 2018, entitled “SYSTEMS AND METHODS FOR GENERATING A MEDICAL NOTE INTERFACE” which is incorporated, in its entirety, herein.

### BACKGROUND

Traditionally, doctors have kept paper, hard copy, records, or notes, documenting their visits, or encounters, with patients. This serves the purpose of documenting aspects of the patient's health and medical condition and may be used to document what services the doctors have performed for their patients, which may be used for medical record keeping and billing purposes. Hard copy records have many disadvantages including requiring the doctor to manually take notes during a conversation with a patient and/or fill out paperwork following an encounter with a patient. At times, these notes may be incomplete and/or difficult to read.

### SUMMARY

Systems, methods, and devices for generating one or more medical note interface(s) are herein described. In some embodiments, responses to a plurality of questions included in a medical questionnaire may be received from a patient and/or on behalf of a patient via, for example, a patient device and/or a user device. The patient and/or user device may be, for example, a smart phone, a tablet computer or a laptop computer. The questions of the medical questionnaire may pertain to, for example a medical condition (e.g., diabetes, heart disease), chief complaint (e.g., headache or shortness of breath), and/or general wellness (e.g., blood pressure, weight, mood, etc.) of a patient. In some instances, the received responses may be responses to an outcome measurement device (OMD), which, in some instances, may be something other than a medical questionnaire. Exemplary OMD responses include, but are not limited to, results for physical examinations, results for diagnostic tests, and reported outcomes. In some embodiments, a medical questionnaire and/or OMD may be a patient reported outcome (PRO) instrument or questionnaire.

The received answers may then be used to prepare a medical note interface, or GUI, that includes the received responses and/or representations of the received responses. Each of the received responses may be correlated to one or more of the questions included in the medical questionnaire. The prepared medical note interface may then be provided to a user via, for example, a display device or screen provided by the device the user is using. At times, the responses may be received prior to a patient encounter with the user and/or a doctor. For example, one or more medical questionnaires may be provided to the patient prior to his or her encounter with the user. In some embodiments, an indication of the time the user spent with the patient during an encounter may be received. The time may be measured by, for example, a timer and/or scheduling information. The time the user spent with the patient during the encounter may be associated with a medical note interface via metadata and/or data displayed on the medical note interface.

In some embodiments, one or more of the received responses may pertain to a chief complaint of the patient and the chief complaint may be added to the medical note interface. In some cases, the chief complaint may be added verbatim to the medical note interface. Additionally, or alternatively, one or more narrative comments regarding the patient may be received from the user and/or the patient. These comments may be to the medical note interface. Exemplary narrative comments include, but are not limited to, the patient's chief medical complaint, an observation of the patient's condition, exam results from a test, an impression, a theory regarding a cause for a patient's medical condition and/or symptom, and a response to treatment.

Additionally, or alternatively, one or more of the received answers may be used to determine a wellness score for the patient. Determination of the wellness score may be facilitated by accessing a scoring procedure for the received responses and/or medical questionnaire and scoring the received responses using the scoring procedure. The scoring procedure may be specific to the medical questionnaire presented to the patient.

In some embodiments, the patient may be associated with an electronic medical record (EMR) and the medical note interface may be associated with the patient's EMR. Additionally, or alternatively, the patient may be associated with a patient account provided and/or managed by a software application running on his or her patient device and the medical note interface may be associated with the patient's account.

In some cases, an indication that the user has reviewed the medical note interface may be received. Exemplary indications include selection of an icon, selection of an option provided by a drop-down menu, and/or entry of text into a text box. Once the indication has been received, the medical note interface may be locked, or otherwise not editable so that the locking prevents modification of the medical note interface. In some cases, locking the medical note interface may include packaging the medical note interface for storage in a blockchain.

In some embodiments, the information associated with one or more medical note interfaces may be used by a medical billing system in order to prepare, for example, a bill for medical services and/or a report showing which medical services were performed for a patient and/or a plurality of patients as may be the case with, for example, an audit. In these embodiments, a database including rules for determining a level of care delivered to the patient during an encounter with the user may be accessed and/or queried and the received responses may be analyzed using the accessed rules in order to, for example, determine a level of care provided to the patient during the encounter responsively. The rules may relate to, for example, the patient's medical history, examination of the patient, medical decision making, time spent with the patient, whether the patient is a new or established patient, patient counseling, user's time spent related to the encounter, and/or coordination of health care for the patient. In some instances, an indication of the level of care may be provided to, for example, the user, the patient, a billing coordinator for the user, and an agency overseeing the user and/or a medical facility with which the user is associated. Additionally, or alternatively, the level of care may be associated with the medical note interface. Additionally, or alternatively, billing information and/or an amount due for payment and/or reimbursement the encounter may be determined responsively to the determined level of care.

In another embodiment, a set of responses to a plurality of questions included in a medical questionnaire may be received from, for example, a patient device and/or a user device. Often times, the questions may pertain to a medical condition and/or state of wellness of a patient. The received responses may then be analyzed using, for example, a scoring procedure associated with the plurality of questions, to determine a wellness score for the patient. A medical note interface that includes the wellness score for the patient may then be generate and provided to the user via, for example, communication of the generated medical note interface to a display device included in, for example, a computer or smart phone.

In some embodiments, an indication that the user has reviewed the medical note interface may be received and the medical note interface may be locked or otherwise made un-editable responsively to receipt of the indication. The locking of the medical note interface may prevent modification of the medical note interface.

In some instances, the received set of responses may be a first set of responses and the determined wellness score may be a first wellness score and a second set of responses to the plurality of questions included in the medical questionnaire may be received. A second wellness score for the patient may then be determined using the second set of received responses. In some embodiments, an improvement score for the patient may then be determined using (e.g., determining a percentage change, subtracting, determining an average percentage change, etc.) the first and second wellness scores. The second wellness score and/or the improvement score may then be added to the medical note interface as, for example, a numerical value and/or a graphically represented value.

At times, a chief complaint of the patient may be received and added to the medical note interface. Additionally, or alternatively, a narrative comment may be received from the user and/or the patient. This narrative comment may then be added (e.g., verbatim, summarized, quantified, etc.) to the medical note interface. Additionally, or alternatively, the patient may be associated with an electronic medical record (EMR) and the medical note interface, received response, improvement score, and/or one or more wellness scores may be associated with the patient's EMR.

### BRIEF DESCRIPTION OF DRAWINGS

The present invention is illustrated by way of example, and not limitation, in the figures of the accompanying drawings in which:

FIGS. 1A and 1B are block diagrams of exemplary systems for generating and/or presenting a medical note interface, in accordance with some embodiments of the present invention;

FIGS. 2A and 2B provide flowcharts illustrating an exemplary process for generating a medical note interface, in accordance with some embodiments of the present invention;

FIG. 3 provides a flowchart illustrating an exemplary process for determining a billing code, in accordance with some embodiments of the present invention;

FIGS. 4A-4N provide screen shots of exemplary medical note interfaces, in accordance with some embodiments of the present invention;

FIGS. 5A-5B provide screen shots of an exemplary overview dashboard interface for a patient account that as may be provided to a patient and/or caregiver, in accordance with some embodiments of the present invention;

FIG. 6A provides a screen shot of an exemplary clinician's view permissions interface, in accordance with some embodiments of the present invention;

FIG. 6B provides a screen shot of an exemplary add a clinician interface, in accordance with some embodiments of the present invention;

FIG. 6C provides a screen shot of an exemplary doctor finder interface, in accordance with some embodiments of the present invention;

FIG. 6D provides a screen shot of an exemplary clinician request interface, in accordance with some embodiments of the present invention;

FIG. 7A provides a screen shot of an exemplary interface by which to add a forum to a patient's feed interface, in accordance with some embodiments of the present invention;

FIG. 7B provides a screen shot of an exemplary patient portal account interface, in accordance with some embodiments of the present invention;

FIG. 7C provides a screen shot an exemplary patient portal account interface showing a search research result, in accordance with some embodiments of the present invention;

FIG. 8A provides a screen shot an exemplary medication details interface, in accordance with some embodiments of the present invention;

FIG. 8B provides a screen shot an exemplary add medication interface, in accordance with some embodiments of the present invention;

FIG. 8C provides a screen shot an exemplary medication note interface, in accordance with some embodiments of the present invention;

FIG. 9A provides a screen shot an exemplary treatment details interface, in accordance with some embodiments of the present invention;

FIG. 9B provides a screen shot an exemplary add treatment interface, in accordance with some embodiments of the present invention;

FIG. 9C provides a screen shot an exemplary add treatment interface, in accordance with some embodiments of the present invention; FIG. 10A provides a screen shot an exemplary medical questionnaire request interface, in accordance with some embodiments of the present invention;

FIG. 10A provides a screen shot an exemplary array medical questionnaires that may be selected interface, in accordance with some embodiments of the present invention;

FIG. 10B provides a screen shot an exemplary selected medical questionnaire interface, in accordance with some embodiments of the present invention;

FIG. 10C provides a screen shot an exemplary medical questionnaire interface, in accordance with some embodiments of the present invention;

FIG. 10D provides a screen shot an exemplary medical questionnaire “share the survey with friend” interface in accordance with some embodiments of the present invention; and

FIG. 11 depicts an exemplary computer system in which computer readable instructions instantiating the methods of the present invention may be stored and executed, in accordance with some embodiments of the present invention.

### WRITTEN DESCRIPTION

Systems, methods, and devices for generating one or more medical note interface(s) are herein described. In many instances, the medical note interfaces disclosed herein may be graphic user interfaces (GUIs). The present invention may be used to, for example, supplement a physician's paper, or hand written, patient notes and, on some occasions, the medical note generated and/or disclosed herein may be printed and placed in a user's (e.g., physician's or hospital's) electronic and/or physical medial files.

Additionally, or alternatively, the present invention may be used to provide information to a patient's electronic medical record (EMR) that may be maintained by, for example, a user, a medical facility, and/or administrative entity, and/or the patient him- or herself. In some instances, the present invention may provide information to and/or extract information from a patient-managed wellness account. In some embodiments, a patient may be able to access and/or input information into one or more of medical note and/or medical note interfaces for him or her and provide information for the note via, for example, answering questions provided to the patient by the user. However, in many cases, the patient may not be able to (e.g., not have permission for) directly enter and/or change information associated with the patient and/or a medical note interface for the patient. Patient-provided information may include, but is not limited to, responses to questions provided by a medical questionnaire, statements of a complaint or chief complaint, comments regarding treatment or a diagnosis, addition of life events or stressors, wellness, symptom management, side effect management, treatment compliance information and so on.

Additionally, or alternatively, the present invention may be used to provide proof of delivery of care in the event of an audit of a user and/or a medical facility associated with the user. In these instances, the medical notes generated via execution of processes described herein may be used to demonstrate that a particular user's encounter with a patient complies with, for example, one or more standards of care and/or recommendations that may be established by, for example, a medical board, a medical association, a medical facility, and/or a treatment provider.

Additionally, or alternatively, the present invention may be used to facilitate the entry of more complex information and/or information of a broader scope into a medical note and/or medical note interface than may be practical during a typical encounter between a patient and a user. For example, it may not be feasible for a user to ask a patient all questions included in a medical questionnaire during an encounter, which may yield incomplete medical care and/or incomplete diagnosis of a problem. Feasibility may be limited due to, for example, time and/or space constraints. In addition, the present invention facilitates communication between caregivers of the patient so that answers to questions that may (or may not) be directly related to a patient's chief complaint may also be considered by the physician when making medical decisions.

Any user of a system, device, and/or method disclosed herein may be referred to herein as a user. Exemplary users include, but are not limited to, medical professionals, medical administrators, doctors, nurses, hospital administrators, clinicians, lab technicians, executive assistants for medical professionals, and patients (i.e., a receiver of medical care).

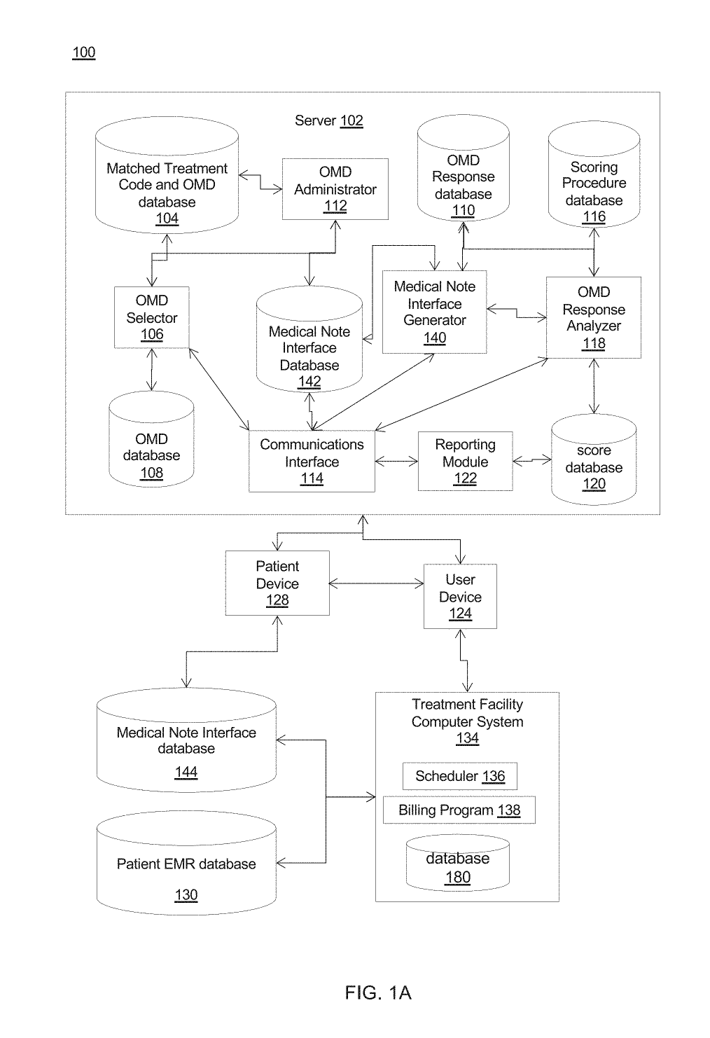

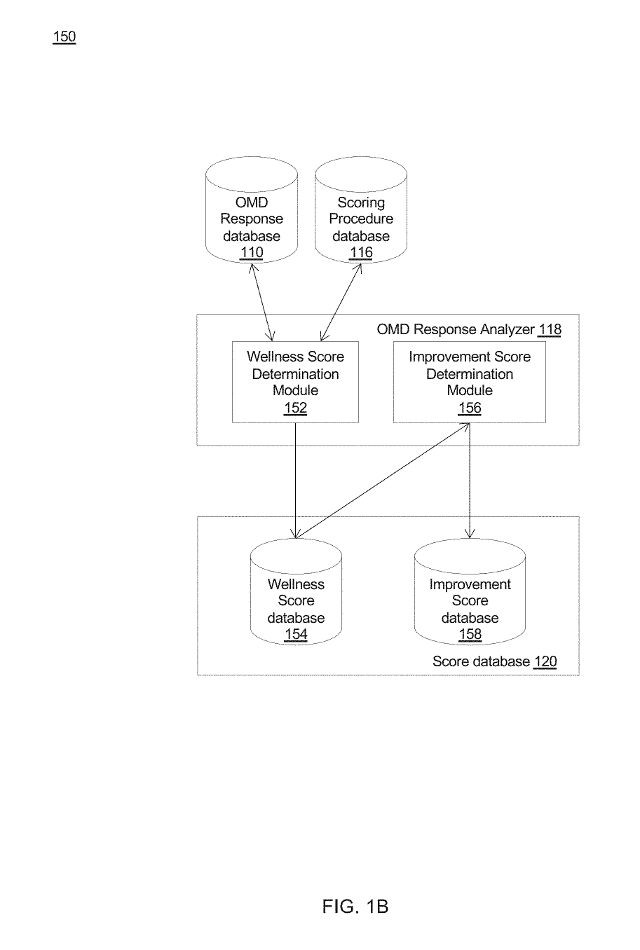

Turning now to the FIGs., FIG. 1A provides a block diagram of an exemplary system 100 that may be used to execute one or more of the processes described herein. At a high level, system 100 includes a server 102, a patient device 128, a user device 124, and a treatment facility computer system 134, all directly or indirectly communicatively coupled to one. Patient device 128 may be any device (e.g., a smartphone, a laptop computer, a tablet computer, a desktop computer, etc.) that enables communication between a patient and other components of system 100. Similarly, user device 124 may be any device (e.g., a smartphone, a tablet computer, a laptop computer, a desktop computer, etc.) that enables communication between a treatment provider (also referred to herein as a “user”) and other components of system 100. In some cases, user device 124 may also be a device that is enabled to perform a specific healthcare treatment and/or diagnostic task. For example, user device 124 may be a network-connected treadmill, a network connected blood pressure monitor, or a network-connected ultrasound machine. For simplicity, only one user device 124 is depicted, while it is understood that in practice there may be a plurality of user devices, one or more for each treatment provider. Similarly, while only one patient device 128 is depicted, it is understood that in practice there may be a plurality of patient devices, one or more for each patient.

Treatment facility computer system 134 may be a computer system that is located in, and/or communicatively coupled to, a treatment facility (i.e., a computer/server that is located in a doctor's office or treatment facility). As is understood in the art, an EMR (as stored in EMR database 130) may include notes prepared by a treatment provider regarding the health of a patient, results of medical tests performed on a patient, treatments administered on a patient, etc. Further due to HIPAA regulations, medical records from treatment facility computer system 134 may be communicated to user device 124, patient device 128 and server 102 using one or more security protocols that may be compliant with HIPAA requirements. It is understood that other data (i.e., not patient-specific data) may be transmitted between user device 124, patient device 128, sever 102 and facility computer system 134 via a conventional communication network (e.g., the Internet, a wired network, a wireless network, a private network, a public network, routers, switches, etc.), which has not been depicted in FIG. 1A. Further, it is understood that user device 124, patient device 128, server 102 and facility computer system 134 may be communicatively coupled to via a communication network (e.g., the Internet) and/or a blockchain.

In one embodiment, any one of the components of system 100 may replace any patient identifying information (e.g., patient name, social security number, birthdate, address, etc.) in medical records with, for example, a binary string to form anonymized medical records containing no patient identifying information (e.g., patient name, social security number, birthdate, address, etc.). More generally, any patient identifying information in medical data (e.g., EMR, questionnaire responses provided by a patient, wellness scores computed for a patient, etc.) may be replaced with a binary string to form anonymized medical data. Such anonymized medical data may be stored at, for example, server 102, treatment facility computer system 134, patient device 128, and/or user device 124, in various databases operated by server 102 (e.g., OMD response database 110, score database 120, etc.), cloud-based storage (e.g., Amazon Web services, Google Cloud platform or Microsoft Azure) (not depicted), etc. In the event the anonymized medical data is intercepted by a malicious individual (e.g., a hacker), patient privacy may still be preserved since the malicious individual will not be able to associate the anonymized medical data with any specific patient.

A mapping between respective binary strings and respective patient identifying information may be securely stored (e.g., stored in an encrypted manner) at one or more components of system 100. Such mapping may enable an electronic device (e.g., server 102, user device 124, and/or patient device 128) to access medical data associated with a specific patient. The steps for an electronic device to access the medical data of a patient may proceed as follows. First, the electronic device may be authenticated by HIPAA compliance server (e.g., the electronic device is required to provide the proper credentials, such as a login identifier and password). Following successful authentication, the electronic device may request medical data concerning an exemplary patient, John Doe. For example, server 102 may map the patient name of “John Doe” to “patient 001010” via the mapping and/or indexing, and the medical data of patient 001010 may be retrieved from a database which stores the anonymized medical data (e.g., OMD response database 110, score database 120, etc.).

OMD and/or medical questionnaire responses received by server 102 may be communicated to a medical note interface generator 140 by, for example, communications interface 114. Medical note interface generator 140 may then generate one or more medical note interfaces by, for example, executing one or more process(es) described herein. Generated medical note interfaces may be stored in, for example, a medical note interface database 142 which may, in some instances, index a patient and/or a patient account to a medical note interface. Additionally, or alternatively, medical note interfaces may be stored in, a medical note interface database 144 that is external to server 102 which may, in some instances, be operated by a treatment facility computer system 134 and/or a user. The medical note interfaces stored on medical note interface 144 may be associated with index a patient, a patient's EMR (stored in patient EMR database 130), and/or a patient account.

In one embodiment, the process flow for system 100 may proceed as follows. Upon server 102 receiving a request from, for example, user device 124 and/or patient device 128, server 102 may provide an outcome measurement device (OMD) to the patient and/or user device 124 and/or 128. An OMD may be a modality, instrument, or tool by which medical information about a patient may be collected. Exemplary OMDs include, but are not limited to, a medical questionnaire, a physical test of the patient (e.g., blood test, physical examination, or blood pressure), and a patient reported outcome (PRO) instrument. At times, an OMD may referred to as a medical questionnaire herein. In some cases, the request to administer the OMD may be triggered via the entry of a treatment code (e.g., a Current Procedural Terminology (CPT) code) or a treatment/diagnostic test name into the patient's EMR (as stored in patient EMR database 130), a treatment facility's billing software (e.g., billing program 138) and/or a treatment facility's scheduling software (e.g., scheduler 136). In some instances, a request to administer and OMD may be triggered by a patient requesting receipt of an OMD via, for example, his or her wellness account and/or a request to administer and OMD may be triggered by a patient who requests to send an OMD to a friend or colleague via, for example, a link to an OMD and/or an invitation to respond to an OMD.

In some instances, when a treatment/diagnostic test name or other related information (other than a treatment/diagnostic code) is received, server 102 may interpret (using, for example, natural language analysis) the treatment/diagnostic test name so that it matches one or more treatment codes. In such cases, OMD selector 106 may determine one or more OMDs that match the treatment code via matched treatment code and OMD database 104. More generally, matched treatment code and OMD database 104 may also include matches between treatment names and OMDs, as well as diagnostic codes and OMDs when selecting OMDs for delivery to a patient device 128 and/or user device 124.

Next, OMD selector 106 may retrieve the one or more determined OMDs from OMD database 108. The retrieved OMDs may be provided to OMD administrator 112, which may administer the OMDs to the patient via, for example, patient device 128 and/or user device 124. In the instance that the retrieved OMDs are patient reported outcome (PRO) instruments, the PRO instruments may be provided to patient device 128. Completed OMDs (also called OMD responses) may be transmitted from patient device 128 and stored in OMD response database 110. More specifically, OMD responses may be stored in OMD response database 110 in an anonymized fashion. For example, OMD responses may be indexed in OMD response database 110 by a binary string, or other anonymous identifier, rather than by a patient name. Similarly, to the discussion above, if an OMD response for a specific patient is desired, the patient name may be mapped to a binary string by, for example, server 102, and the OMD response associated with that binary string may be retrieved from OMD response database 110.

OMD response analyzer 118 may analyze the OMD responses stored in OMD response database 110 to generate one or more scores (e.g., a wellness score, an improvement score, etc.). Such scores are described in more detail below with regard to FIG. 1B. Such analysis may rely upon scoring procedures stored in scoring procedure database 116. Such scoring may also rely upon other considerations and/or esoteric factors 132 stored at patient EMR database 130. In most circumstances, what may be referred to herein as “other considerations” are factors that may directly, or closely, relate to and/or have an impact on, a medical condition, diagnosis, and/or treatment. For example, it is known that smoking has an impact on a person's cardiovascular health. Thus, whether a person smokes may be an “other consideration” for a patient's treatment related to cardiovascular health. This relationship between cardiovascular health and smoking may be indexed or otherwise stored in other consideration database 132. An esoteric factor is one that is less directly related to a medical condition, diagnosis, and/or treatment but may still have an impact thereon. For example, a vegetarian diet may have an impact on a person's cardiovascular health, yet this impact may be less well understood when compared with the impact of smoking on the same patient's cardiovascular health. As such, a person's status as a vegetarian may be considered an “esoteric factor.”

The scores that are generated by OMD response analyzer 118 may be stored at score database 120. More specifically, scores may be stored in score database 120 in an anonymized fashion so as to, for example, comply with HIPAA regulations or other data security requirements/preferences. For instance, wellness scores associated with a patient may be indexed by a binary string in score database 120 rather than by a patient name.

Finally, reporting module 122 may report the scores to one or more of user device 124, patient device 128 and treatment facility computer system 134. In addition to the request for a treatment, there are other events that may prompt an OMD to be administered to a patient. In one example, the scheduling of an initial appointment (e.g., a consultation) for a patient to discuss a medical condition with a healthcare professional may prompt an OMD to be administered to the patient. Administering an OMD to the patient prior to this initial appointment may be useful for establishing a baseline state of health for the patient, but the selection of the OMD may have some complexity, as no treatment code, treatment name or diagnostic code may yet be available when the initial appointment is scheduled. In many instances, all that the patient will provide is a brief description of the symptoms he/she may be experiencing (e.g., shortness of breath, fever, etc.) and/or a chief complaint. In one embodiment, such symptoms may be provided to OMD selector 106, which attempts to match the symptoms with one or more treatment codes, treatment names, or diagnostic codes.

Such matching by OMD selector 106 may be performed using a learning machine. For instance, matches between, for example, symptoms and treatment codes; symptom and treatment names; and/or symptoms and diagnostic codes) may be provided by a healthcare professional when treating patients, and such matches may be used to train a model that can then be used to determine treatment codes, treatment names or diagnostic codes based on, for example, a patient's symptoms and/or treatment provider notes. Upon determining a treatment code, treatment name, or a diagnostic code, OMD selector 106 may select one or more OMDs based on matches provided in matched treatment code and OMD database 104 (as described above). It is anticipated that the determination of a treatment code, treatment name or diagnostic code by OMD selector 106 may be, in some instances, an imperfect process, so a healthcare provider, or other expert, may be asked to make any necessary adjustments to the treatment code, treatment name and/or diagnostic code determination, before OMD selector 106 selects the one or more OMDs.

As another example, the billing for a medical appointment during which a patient discusses a medical condition with a healthcare professional may prompt an OMD to be administered to the patient. More specifically, billing program 138 may notify server 102 when a bill (or invoice) is generated. If the billing for the appointment occurs after the appointment has concluded, the bill may be associated with a diagnostic code (which may be determined by the healthcare professional during the medical appointment). OMD selector 106 may use the diagnostic code to locate one or more appropriate OMDs for the patient with the assistance of matched treatment code and OMD database 104.

In the examples provided above, it was assumed that an OMD is administered to a patient via patient device 128. In other instances, a medical professional may be required to administer the OMD to the patient. For example, server 102 may notify user device 124 that one or more OMDs should be administered as part of, for example, a medical examination of a patient. In one example, if a patient has recently undergone cardiothoracic surgery, OMD administrator 112 may provide one or more OMDs to user device 124 (e.g., the Intrathoracic Gas Volume Test, Total Lung Capacity Test, Vital Capacity Test, 6 Minute Walk Test, Aortic Insufficiency Test, Mitral Regurgitation Test and/or Aortic Valve Area Test) that could, or should, be administered to the patient during an exam and/or provide one or more mechanisms to user device 124 (e.g., fillable forms) for the treatment provider to enter the OMD responses.

FIG. 1B depicts one embodiment of a system 150 that supports the operation of OMD response analyzer 118 and score database 120 (and some associated components). OMD response analyzer 118 may comprise wellness score determination module 152. In one embodiment, wellness score determination module 152 retrieves responses to an OMD from OMD response database 110, and further may retrieve a scoring procedure associated with the OMD responses from scoring procedure database 116. The scoring procedures may be indexed by, for example, an identifier of an OMD, for which responses have been received, making for easy retrieval of a corresponding scoring procedure. Various scoring procedures may be employed to score a completed OMD, and in one embodiment, the generated score may be known as a “wellness score”. In some cases, a “wellness score” may serve to indicate how severe a patient's symptoms are. In these cases, a low wellness score may indicate that a patient's symptoms are relatively more severe than a higher wellness score such that a subsequent higher wellness score indicates an improvement (i.e., decrease in severity) in the symptoms.

In the case where an OMD is a questionnaire (or PRO instrument), a certain weighing may be used to score or evaluate the patient's responses. For example, certain responses that are more objective in nature (e.g., heart rate, blood glucose level, etc.), may receive greater weights (and hence have a greater influence on the wellness score) than certain responses that are more subjective in nature (e.g., degree of pain, mood, etc.). The reverse scenario, of course, could be true in which subjective responses receive a greater weight than objective responses (e.g., fatigue or mental illness). Scores generated by wellness score determination module 152 may be stored in wellness score database 154. The wellness scores may be indexed in various fashions, for ease of retrieval. In one embodiment, wellness scores may be indexed according to one or more of a patient identifier (e.g., binary string to protect patient privacy), medical condition, treatment provider, treatment facility, time at which OMD was completed, etc.

Improvement score determination module 156 may retrieve two wellness scores for a patient (e.g., a first score calculated for an OMD completed at a first time point and a second score calculated for an OMD completed at a second time point) from wellness score database 154. Improvement score determination module 156 may calculate the difference between the first and second score, and such difference may be known as an improvement score. The improvement score may be stored in improvement score database 158. In one refinement, a relative improvement score may be calculated as the improvement score (i.e., the difference described above) normalized by a maximum improvement score, which may be calculated based on, for example, other considerations 132 stored in a patient's EMR. The maximum improvement score may take into consideration other factors such as the state of a patient prior to a medical treatment (e.g., if patient was in fairly good health, the maximum improvement score might be lower than if the patient was in poor health), and/or the age of a patient (e.g., younger patients might have a higher maximum improvement score than older patients), etc. An improvement score (or a relative improvement score) may be stored in improvement score database 158. The improvement scores may be indexed in various fashions, for ease of retrieval. In one embodiment, improvement scores may be indexed according to one or more of a patient identifier (e.g., binary string to protect patient privacy), medical condition, treatment provider, treatment facility, and time duration over which improvement score was measured, etc.

The components and/or databases of systems 100, 150, and/or 103 of FIGS. 1A, 1B, and/or 1C may be a series of one or more components (e.g., computers, servers, databases, etc.) that may, in some instances, be geographically disparate.

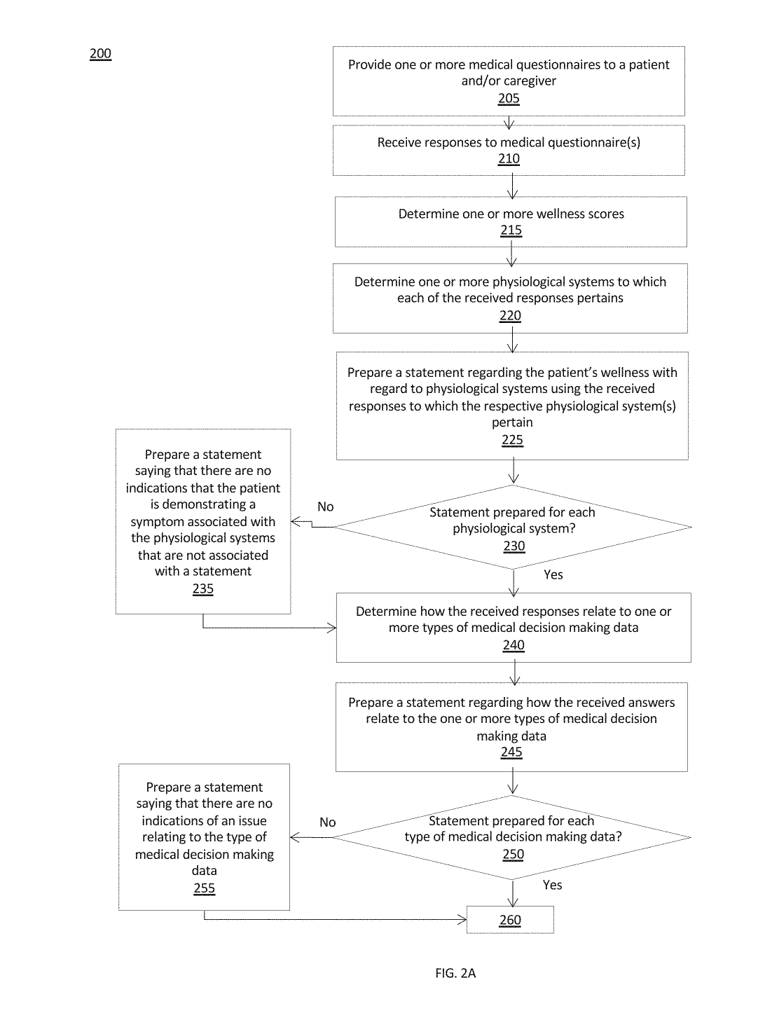

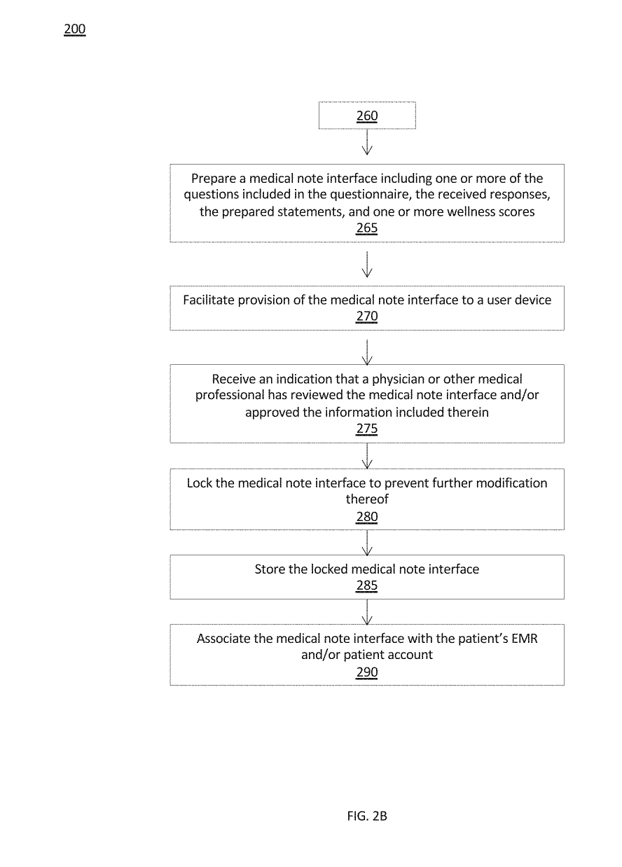

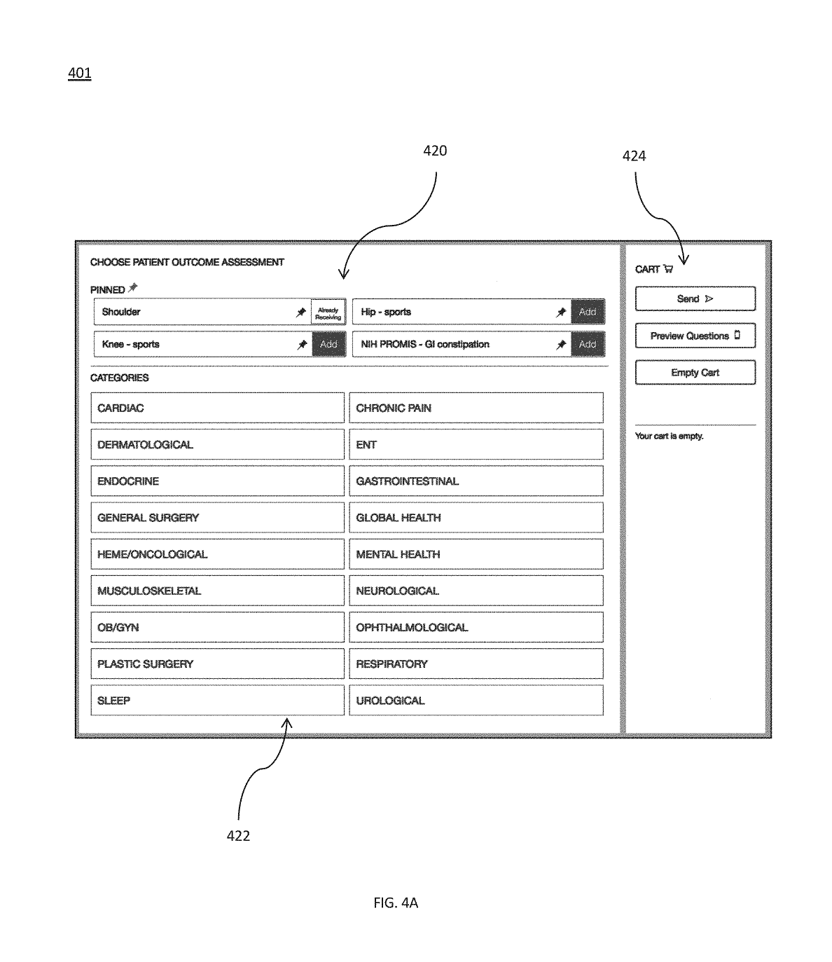

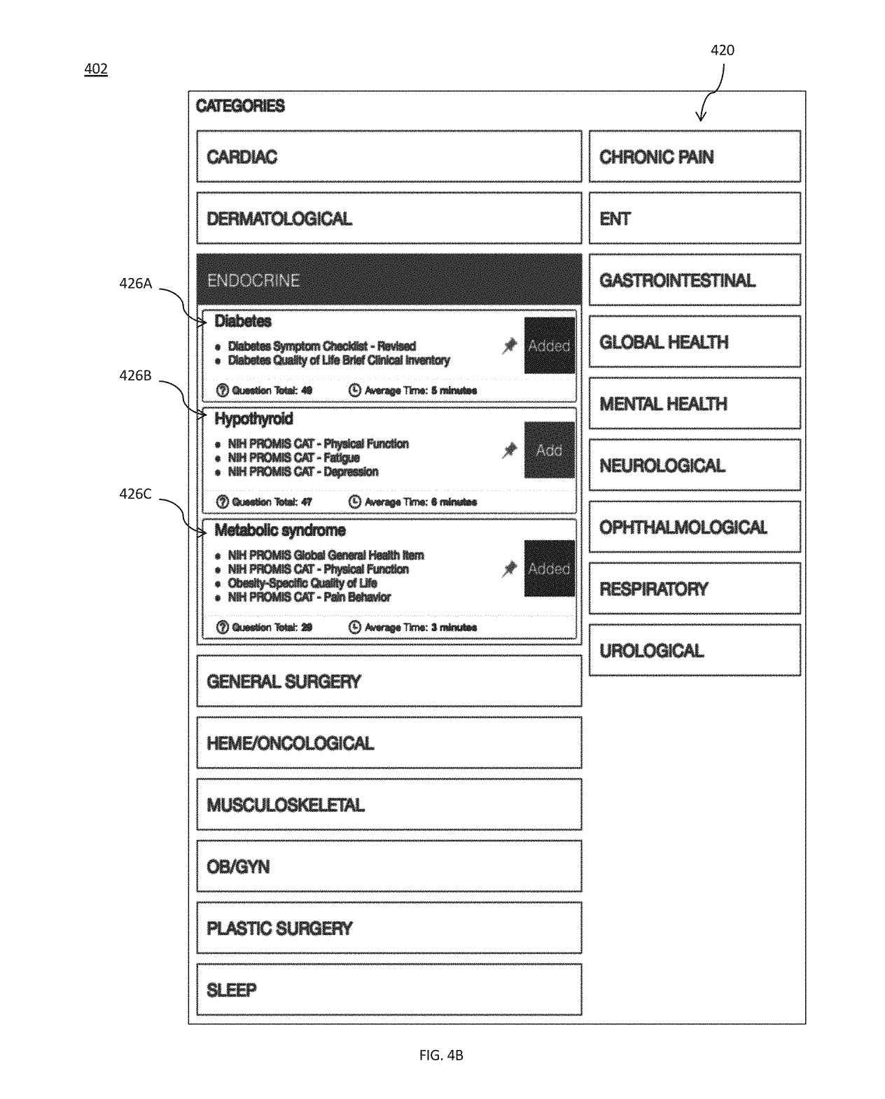

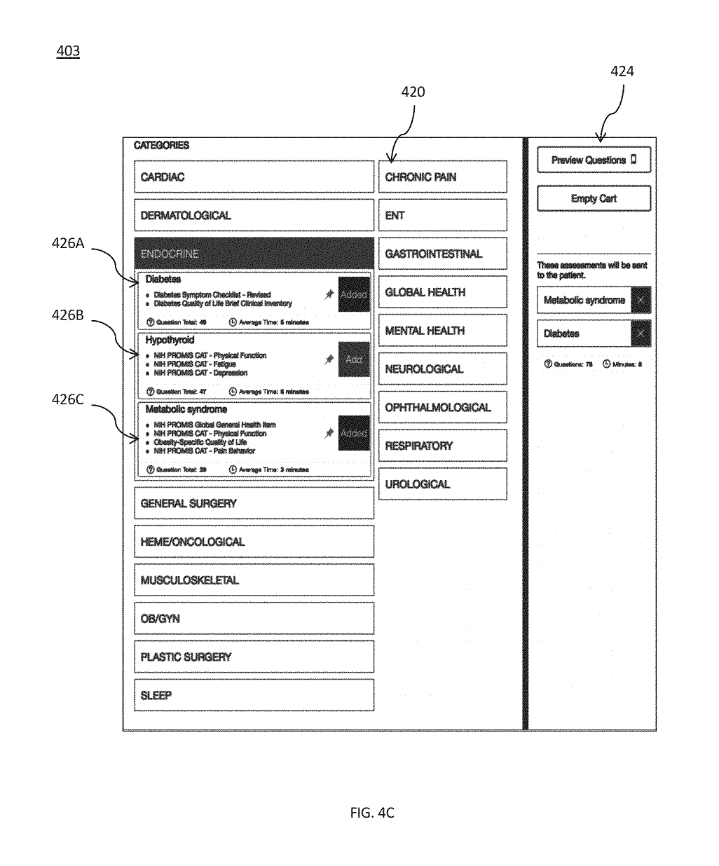

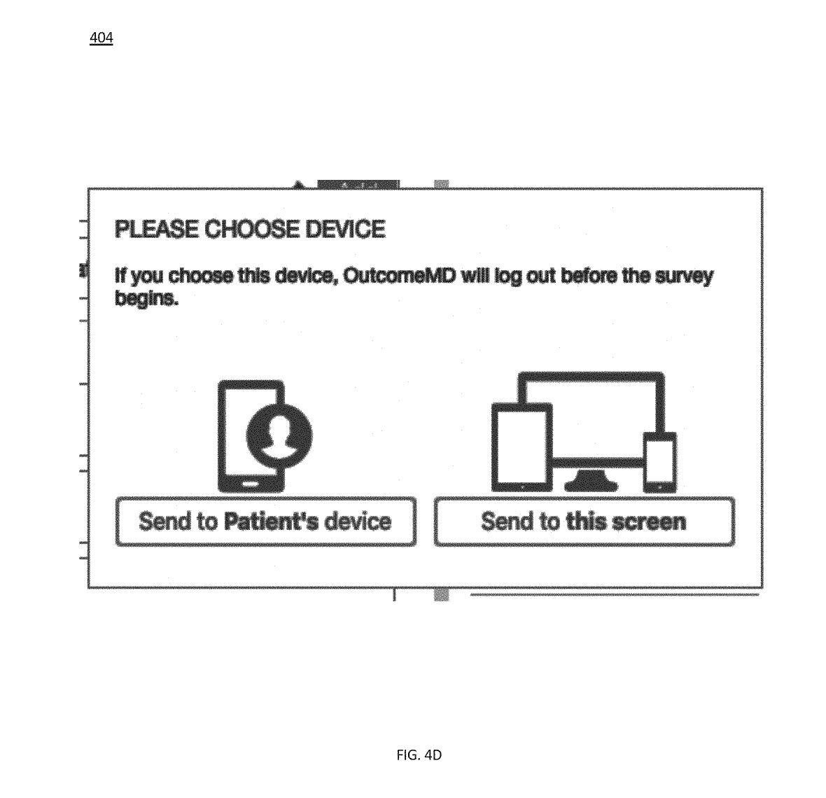

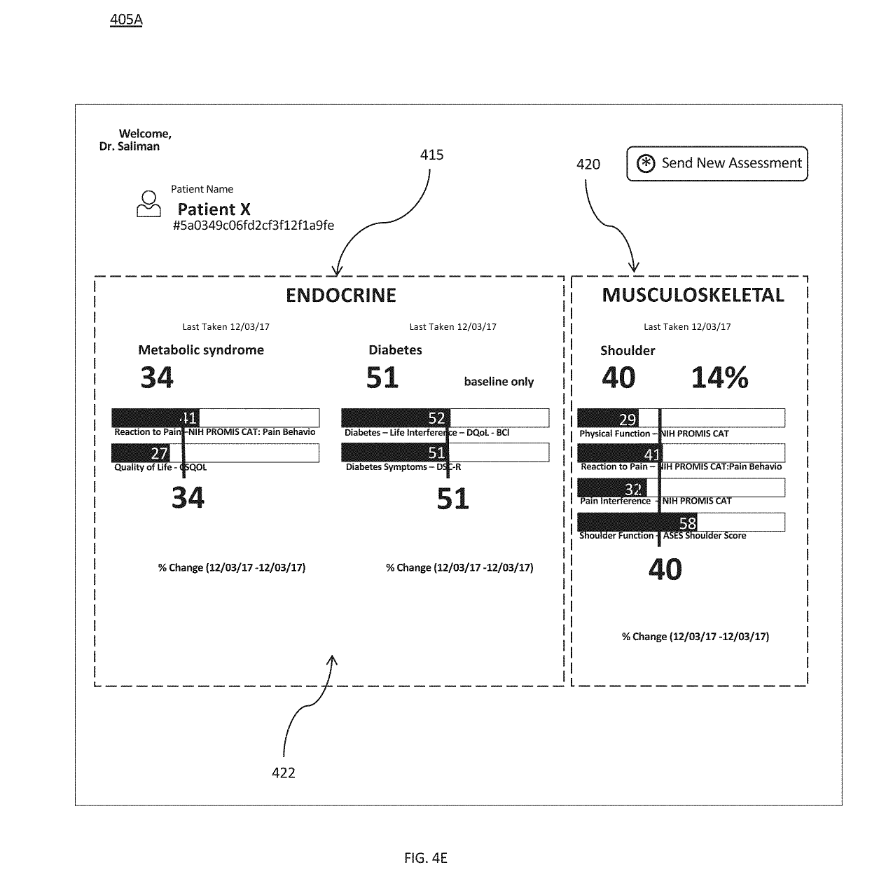

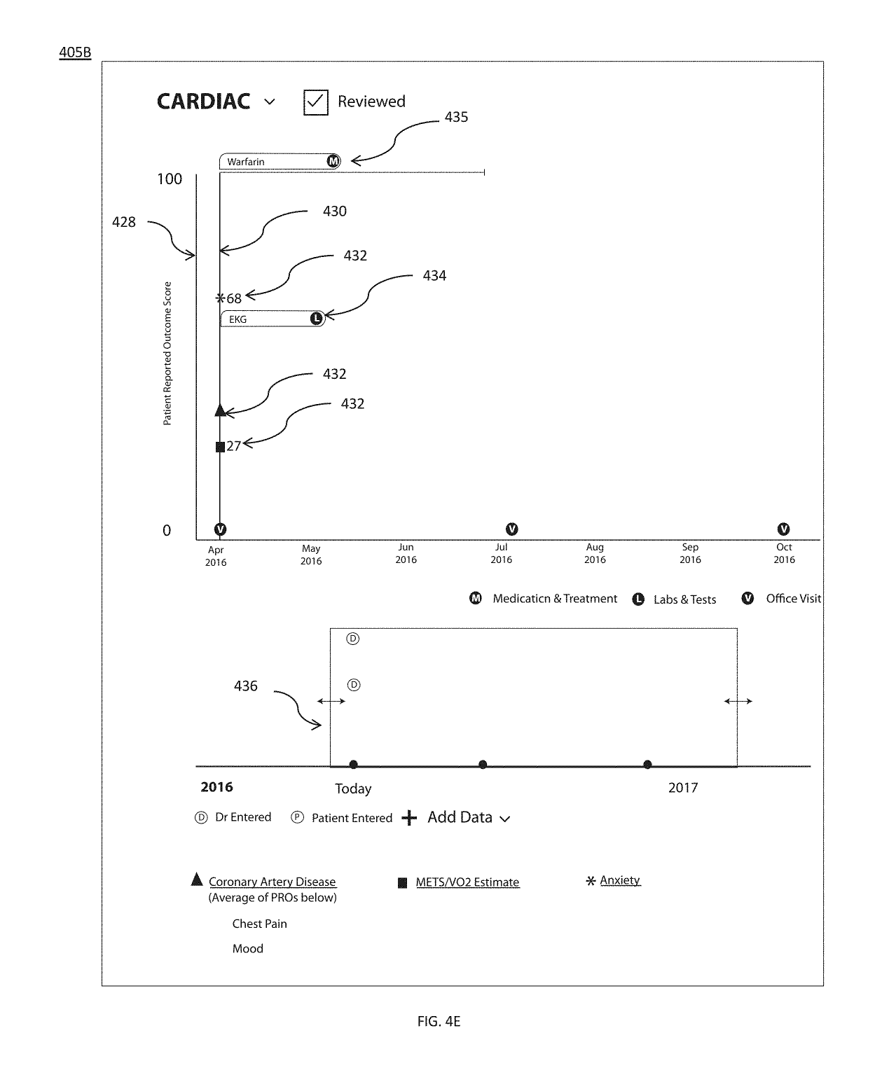

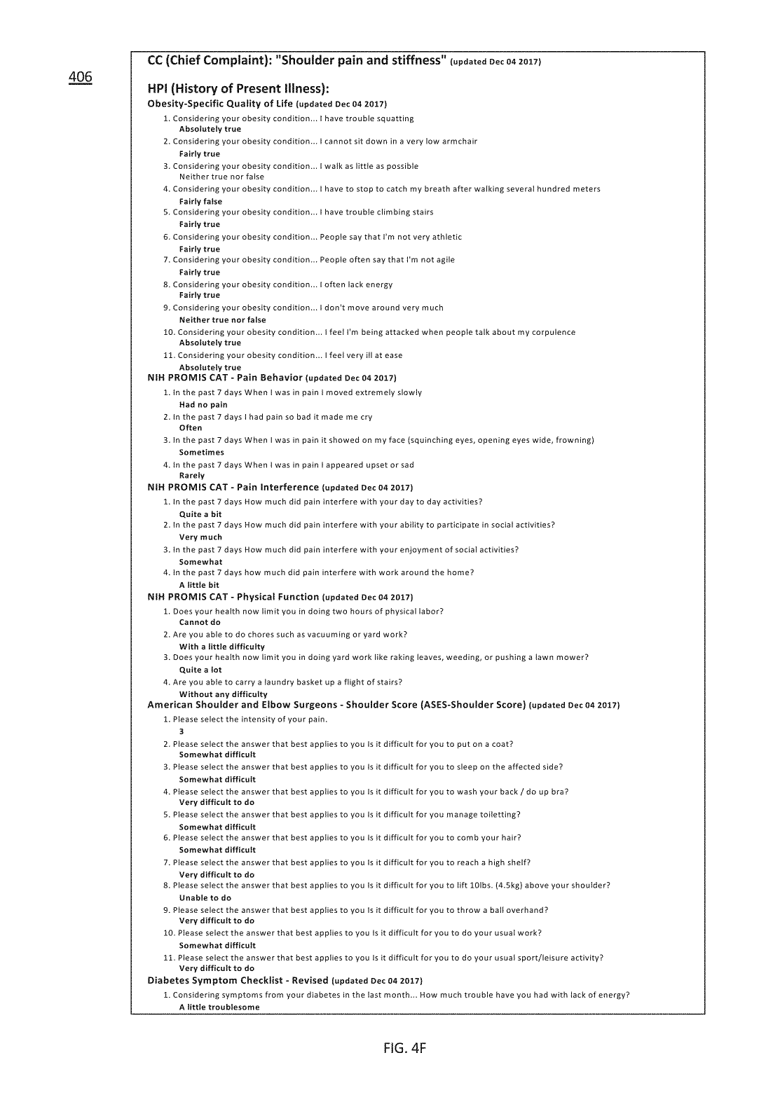

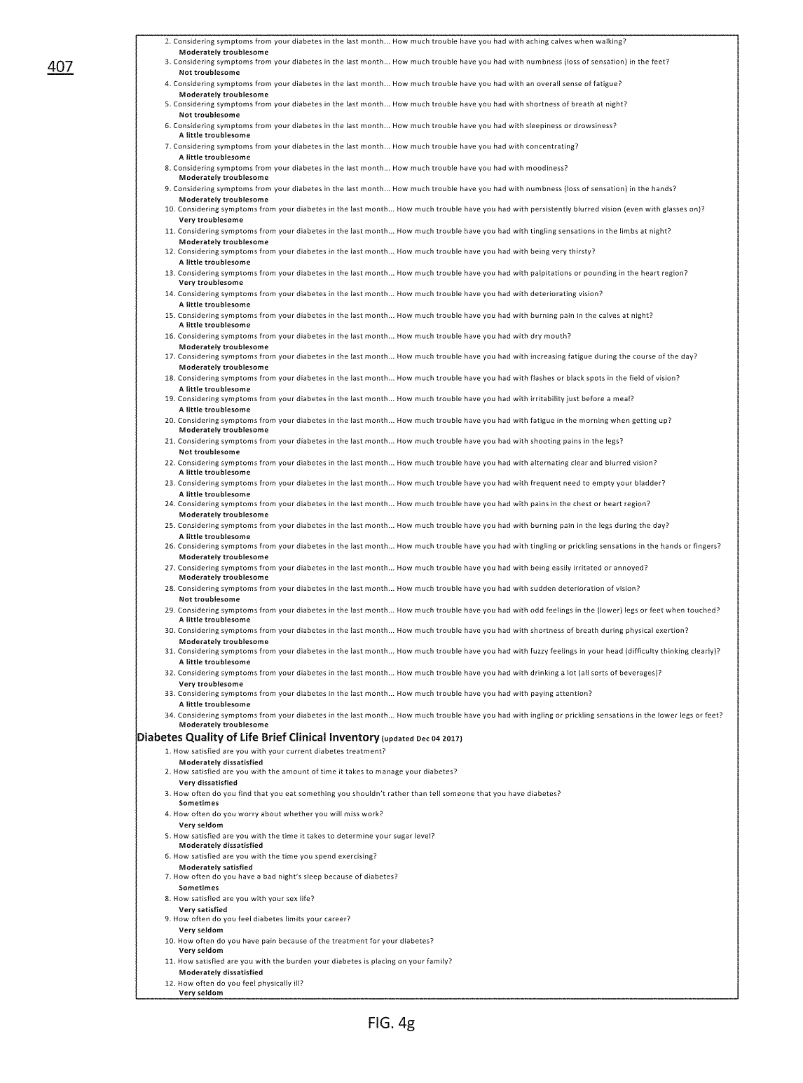

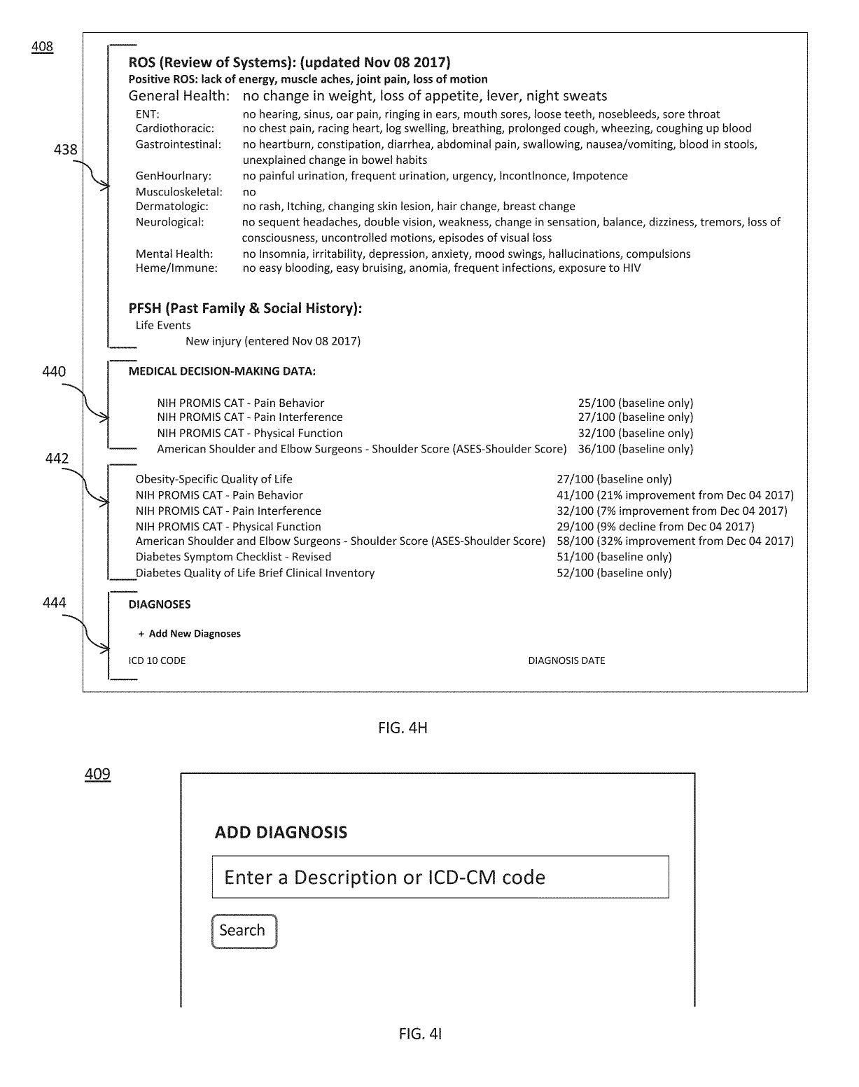

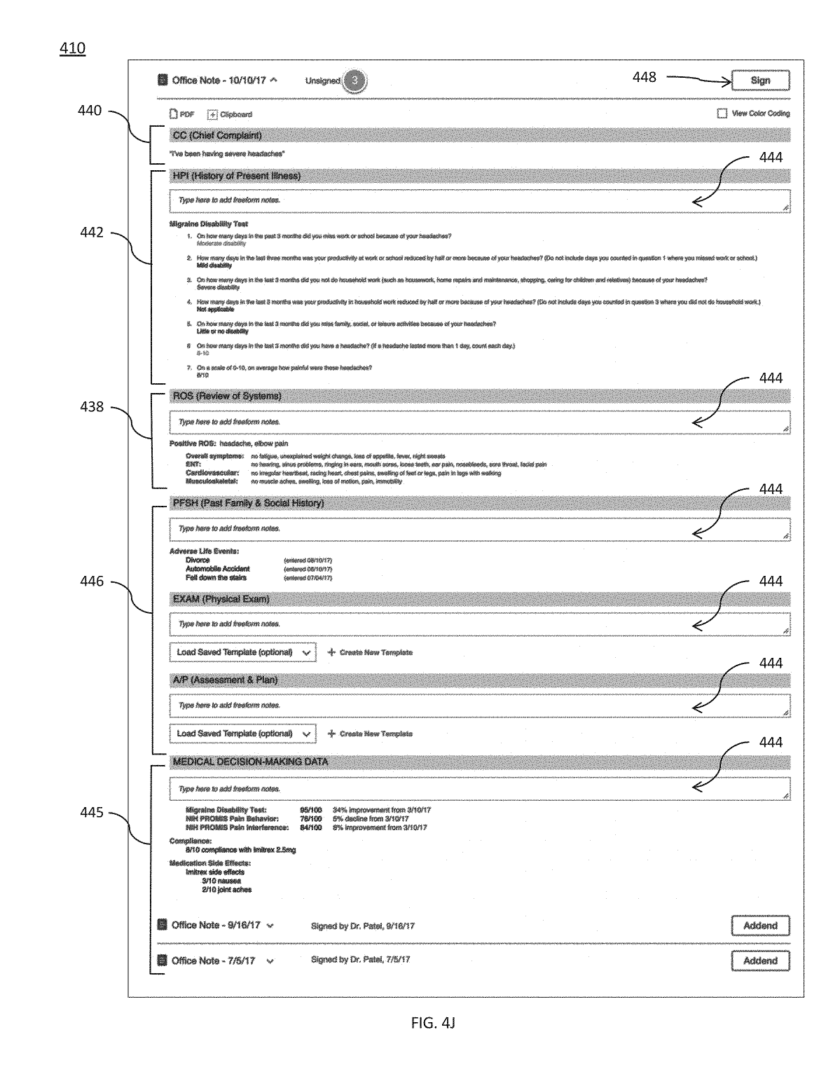

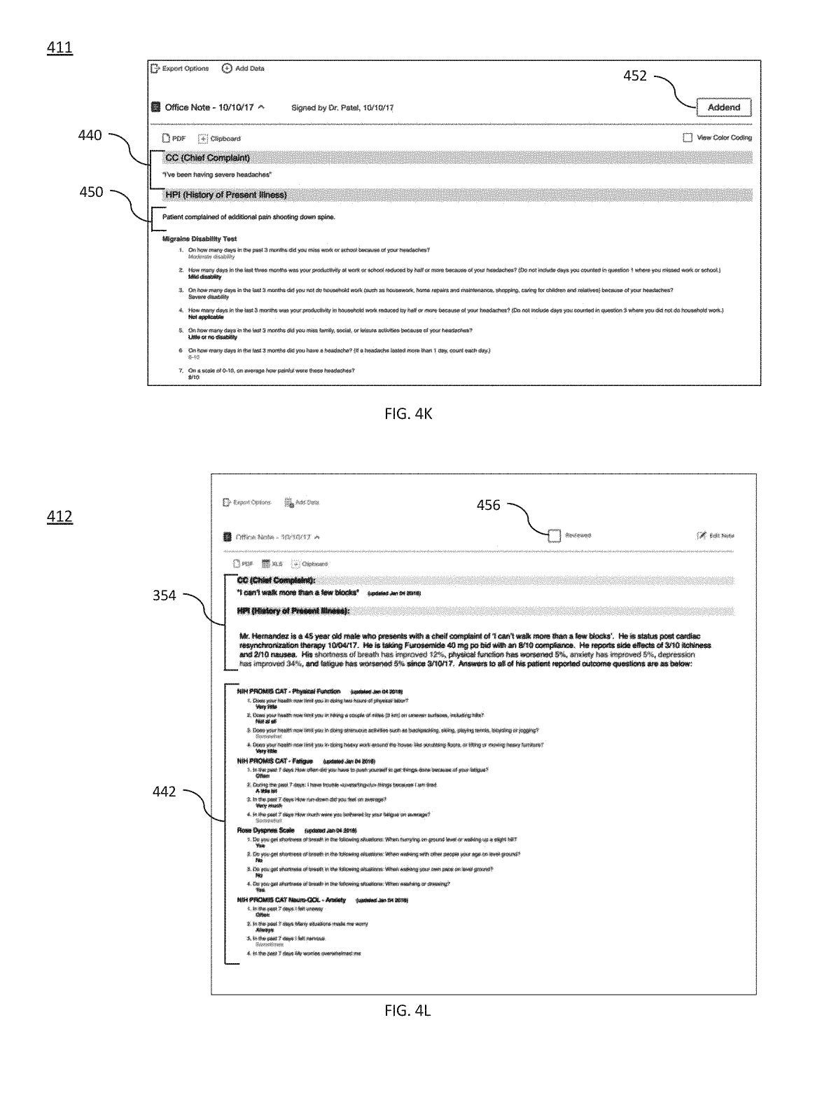

FIGS. 2A and 2B provide a flowchart illustrating exemplary steps of a process 200 for generating a medical note interface, which in some instances may be a graphic user interface (GUI). Process 200 may be executed by, for example, system(s) 100, 150, and/or 1100, and/or portions thereof. Additionally, or alternatively, process 200 may be executed by a server in communication (via, e.g., the Internet and/or a secure network) with, for example, a patient's electronic device, an electronic device operated by a medical facility and/or medical facility computer system. Execution of process 200 may generate a medical note interface, examples of which are provided in the screen shots of medical note interfaces 401-412 of FIGS. 4A-4L, respectively, which will be discussed concurrently with steps of process 200 used to generate respective portions/sections of a medical note interface.

Initially, in step 205, one or more OMDs and/or medical questionnaires (OMDs and medical questionnaires may be collectively referred to herein as a “medical questionnaire”) may be provided to an electronic device (e.g., computer, tablet, mobile smart phone, etc.), such as patient device 128 by which a patient (or a caregiver of the patient (e.g., a family member or friend)) may view the medical questionnaires and provide responses thereto. In some instances, the electronic device is owned and/or operated by the patient or caregiver as their personal electronic device. In other instances (e.g., when the patient or caregiver does not have a personal electronic device), the one or more medical questionnaires may be provided to an electronic device operated by, for example, a medical facility or physician. Step 205 may be executed when, for example, the patient requests a visit with his or her physician as a way to gather information about, for example, the patient's primary complaint or medical concern, the patient's general state of health, and/or the patient's wellness with regard to one or more medical conditions.

FIG. 4A provides a screenshot of an exemplary medical questionnaire selection interface 401 that may be provided to a clinician prior to execution of step 205. Interface 401 includes an array of four pinned icons 420 (hereinafter called a “pinned array 420”) that represent medical questionnaires of relevance to a patient and an array of icons 422 (hereinafter called a “category array 422”) that represent different categories of medical questionnaires that may be selected. In some embodiments, the icons of pinned array 420 may represent pre-selected medical questionnaires and/or recommended. The pre-selection and/or recommendation of medical questionnaires may be based on a number of factors including, but not limited to, a patient's medical history, a chief complaint of the patient, and/or medical questionnaires that have been sent to the patient in the past. For example, pinned array 420 shows that the shoulder questionnaire is one that the patient is already receiving, and the knee-sports, hip-sports, and NIH PROMIS-GI constipation are recommended, or suggested, questionnaires for the patient. A medical questionnaire shown in pinned array 420 may be sent to the patient by selecting the “Add” icon as will be discussed in greater detail below.

Medical questionnaire selection interface 401 also includes a menu 424 that shows a cart icon, a send button, a preview questions button, and an empty cart button. Selection of the cart button triggers display of a list of medical questionnaires the user has selected to send to the patient. Selection of the preview questions button may trigger a display of one or more questions included within a selected medical questionnaire, and selection of empty cart button may trigger the deletion of one or more selected medical questionnaire from the cart.

Category array 422 may include an array of icons that represent medical questionnaire categories, which may correspond to, for example, physiological systems, types of activities, and/or symptoms. In some embodiments, icons of category array 422 may relate to medical questionnaire categories specific to the user. For example, if the user is an orthopedic surgeon or a cardiologist, the medical questionnaire categories included in category array 422 may pertain to categories specific to the respective medical specialty.

In some instances, selection of a medical questionnaire category icon within category array 422 may trigger display of an icon 426 representing one or more sub-categories and/or medical questionnaires associated with the medical questionnaire category as shown in medical questionnaire interface 402 provided by FIG. 4B. For example, when the medical questionnaire category icon associated with endocrine is selected, three icons 426A, 426B, and 426C which represent three different medical questionnaires (in this case diabetes, hypothyroid, and metabolic syndrome, respectively) may be displayed. The user may select one or more of the medical questionnaires in the endocrine category by selecting, or otherwise activating, the “add” button corresponding to the desired medical questionnaire of icon(s) 426A, 426B, and/or 426C. Once the “add” button for a particular medical questionnaire is selected, the selected medical questionnaire may be added to a queue of medical questionnaires to be provided to a patient. In some instances, the medical questionnaires in this queue may correspond to the medical questionnaires received in step 205.

In the example of medical questionnaire interface 403 provided by FIG. 4C, the diabetes and metabolic syndrome medical questionnaires have been added to a queue of medical questionnaires to be provided to a patient and are displayed in a list of assessments/medical questionnaires to be sent to the patient provide by menu 424. Additionally, the “add” button for diabetes and metabolic syndrome shown in interface 402 has changed to an “added” button for diabetes and metabolic syndrome. Should the user wish to de-select a medical questionnaire from the list to be provided to the patient, he or she may select the corresponding “added” icon. The list of medical questionnaires provided by interface 403 may also be provided responsively to selection of the cart icon show in interface 401.

Once the user has selected one or more medical questionnaires to provide to a patient, he or she may select the send icon of menu 424, which may trigger display of an interface like device selection interface 404 provided by FIG. 4D. Device selection interface 404 provides a user with two options for communication of one or more medical questionnaires: send to patient's device and send to this screen, the selection of one may enable communication of the selected medical questionnaire(s) to the selected device for completion by the patient.

In some cases, step 205 may be executed via, for example, sending a hyperlink, GUI, or other enriched media document (e.g., a fillable form) to the patient's electronic device via, for example, a short message service (SMS) text message or email. Additionally, or alternatively, a medical questionnaire may be added to a patient's account—to be accessed via his, or her, personal electronic device at another time. In most instances, the format by which the medical questionnaires are sent to the patient will enable the patient to directly communicate his or her responses to the questions of the questionnaire to a server or other computer system executing process 200 or a portion thereof without the need of additional data entry by, for example, a medical professional or other medical office staff.

In step 210, responses to the one or more medical questionnaires may be received via, for example, direct entry of responses into an interface, or GUI, provided in step 205. The responses may be received from, for example, a patient device like patient device 228 and/or a treatment provider device like treatment provider device 224. FIGS. 4F and 4G [NEED TO RENUMBER] provide exemplary portions of a medical note interface 406 and 407, respectively, that provide an example of questions of a medical questionnaire that may be provided to a patient in step 205 and answers to those questions that may be received in step 210 that are formatted into an exemplary medical note interface 406 and 407 so that they may be reviewed by the user. In addition to responses to questions of a medical questionnaire, the responses received in step 210 may include an indication of the patient's chief medical complaint as articulated by the patient (in most cases, verbatim) along with a reproduction of the questions asked by the various questions of each medical questionnaire (organized according to sub-medical questionnaire) provided to the patients along with corresponding answers received from the patient. These interfaces provide a way for the patient to directly enter (via their responses to questionnaires) information into a medical note interface for review by the user and/or entry into the patient's electronic medical record. The categories and/or medical questionnaires selected by the user via, for example, interaction with an interface like interfaces 401, 402, 403, and/or 404 may be represented by the questions and responses provided by interfaces 406 and 407. For example, interface 406 indicates that the Obesity-Specific Quality of Life, NIH PROMIS CAT—Pain Behavior, NIH PROMIS CAT—Pain Interference, NIH PROMIS CAT—Physical Function, American Shoulder and Elbow Surgeons-Shoulder Score (ASES-Shoulder Score), and Diabetes Quality of Life Brief Clinical Inventory medical questionnaires were selected by the user for provision to the patient prior to execution of step 205.

Optionally, a wellness score and/or an improvement score for one or more aspects of the patient's medical condition and/or physiological systems may then be determined (step 215). In some embodiments, determination of a wellness score may include querying a scoring database like scoring procedure database 216, for a scoring metric and/or scoring procedure associated with the medical questionnaire provided in step 205. In some instances, this querying may include retrieving a scoring procedure from scoring procedure database 216 using an identifier of the medical questionnaire. For instance, a medical questionnaire may be associated with a code (e.g., 3232) and this code may be used to retrieve a scoring procedure from scoring procedure database 216. Example scoring procedures include taking an average of all the patient responses (e.g., assuming all responses are numeric), taking a weighted average of the patient responses (e.g., weighting certain responses higher than other responses), adjusting the range of patient responses (e.g., changing responses choices from 2, 3, 3 to 1, 4, 6). In some embodiments, execution of step 215 also includes retrieval of a sub-scoring procedure that may be specific to the patient (i.e., associated with the patient's account or a co-morbidity of the patient) as may be indicated by, for example, the patient's wellness account and/or EMR. The scored responses may then be used to determine a wellness score associated with the received response and/or a sub-set of received responses.

An improvement score may be a determination of how a patient's condition has changed over time. In some embodiments, determination of an improvement score may involve comparing (e.g., averaging, subtracting, determining a percentage change, determining a time weighted average, etc.) one or more previously determined wellness scores with a currently determined wellness score in order to determine how a patient's wellness score has changed over time (e.g., 3 weeks, 3 months, 1 year, etc.).

FIG. 4E1 provides an exemplary wellness score portion of a medical note interface 405A for a patient who has provided answers to the medical questionnaires for metabolic syndrome and diabetes within the endocrine category/physiological system as shown in first category/physiological system window 415 and shoulder, which falls into the musculoskeletal category/physiological system as shown in second category/physiological system window 420 and provides corresponding wellness score information. More specifically, first category/physiological system window 415 provides an overall wellness score for the metabolic syndrome medical questionnaire of 34 along with wellness scores for each of the sub-medical questionnaires (in this case, a reaction to pain—NIH PROMIS CAT Pain Behavior wellness score of 41 and a quality of life wellness score of 37) included in the metabolic syndrome medical questionnaire and an overall wellness score for the diabetes medical questionnaire of 51 along with wellness scores for each of the sub-medical questionnaires included in the diabetes medical questionnaire (in this case, a diabetes life interference DQoL—BCI wellness score of 52 and a diabetes symptoms wellness score of 51). Both of these wellness scores are baseline only because they have not been determined for patient before. The second category/physiological system window 420 shows an overall wellness score of 40 for the shoulder medical questionnaire along with wellness scores for the four sub-medical questionnaires included in the shoulder medical questionnaire. Second category/physiological system window 420 also shows an improvement score of 14% along with an upward pointing arrow, which represents a change (in this case a wellness score that has increased by 14%) between the one or more previously determined wellness scores for the shoulder category. The improvement score may be determined by, for example, comparing previously determined and currently determined wellness scores. Were the improvement score to show a decline between a present and one or more previously determined shoulder wellness scores, the % change/improvement score may be accompanied with an indicator of the decline such as a downward pointing arrow or a different color (e.g., red). In some instances, the wellness scores may be determined for different aspects of the patient's wellness regarding symptoms, diagnoses, or other aspects associated with, for example, a physiological system, chief complaint, diagnosis, and other factors that may contribute to medical decision making (i.e., may be medical decision-making data) for the patient. In some instances, the wellness score interface 405A may also display changes in wellness scores, or improvement scores, determined for the patient over time.

FIG. 4E2 provides another exemplary medical note interface 405B for a patient (in this case, Isaac Hernandez) pertaining to the patient's cardiac wellness. An upper portion of medical note interface 405B includes a graph 428 that plots time along the Y-axis and wellness score (from −100) on the X-axis. In some embodiments, the scales of the X- and Y-axis(es) may be different. Graph 428 also shows an event line 432 corresponding to a date on which the patient visited, or had an encounter with, the user. The event line is a straight line along the Y-axis that corresponds in position to when the patient had the office visit/encounter with the user. The event line 432 of interface 405B shows a series of 3 different icons 432 that represent three different wellness scores determined for the patient during an office visit and/or encounter with the user in April 3017 and responsively to responses received from the patient in, for example, step 210. Wellness score icons 432 may be of any appropriate shape, color, and/or size and may coded be (e.g., color, shape, size, etc.) so that each wellness score provided on the line is easily distinguished from one another when displayed. In some cases, a wellness score icon 432 may be hyperlinked to further information regarding, for example, the information used to determine the subject wellness score.

Graph 428 also includes action icons 434 that provide an indication of an action taken by the user. Actions represented by an action icon 434 include, but are not limited to, prescription of a medication, requesting of a test, and/or prescription of a physical therapy regimen, dietary change, exercise regimen etc. In the example of interface 405B, the action icons 434 represent medication prescription for Warfarin and a lab test for an EKG. In some embodiments, one or more action icons 434 may include an indication of the type of action they represent. Exemplary indications include, but are not limited to, a label (e.g., M for medication, L for lab test, etc.), a color, and/or a position along event line 430 (e.g., lab tests positioned below medications on event line 430). In the example of Interface 405B, the action icon 434 for Warfarin it Is labeled with an M to represent that it is a prescribed medication and the action icon for an EKG is labeled with an L to represent that it is a prescribed a lab test. In some embodiments, and action icons 434 may be accompanied by a visual indication of a duration the action item applies to the patient. For example, the Warfarin action icon 434 of interface 405B is accompanied by a timeline that extends from the event line 430 through July 3016 thereby indicating that the patient has been prescribed Warfarin fourth the interval between April 3016 and July 3016.

In some embodiments, one or more of the graphic elements/icons provided by interface 405B may be linked to other information (e.g., notes regarding the event, patient's medical history, details of a prescribed action item, etc.) and the may access this information by, for example, selection of one or more of the graphic elements/icons provided by interface 405B. At times, display of the wellness score(s) and/or icons may be responsive to a selection of an icon or other provided instruction. For example, selection of the office visit (V) icon may populate the graph with a series of vertical lines that indicate when, along the timeline of the graph, office visits occurred; selection of a medication & treatment (M) icon may populate the graph with action icons 434 representing medications and/or treatments prescribed to the patient in a manner that may, or may not be, aligned with office visits; and selection of a labs & tests (L) icon may populate the graph with an indication of a lab and/or test prescribed to/performed on or for the patient in a manner that may, or may not be, aligned with office visits.

A graphic display 436 on the lower portion of medical note interface 405B may provide a graphic display of when and what type of information is entered into, for example, the patient's wellness account and/or her, which may correspond to information provided by graph 428. For example, the graph of medical note interface 405B provides two blue dots that provide an indication that the doctor, or user, has entered a note or otherwise altered or added to an existing medical note for the patient into, for example, the patient's wellness account and/or EHR. The two blue dots of graphic display 436 represent the action icons 434 of graph 428.

Then, one or more physiological system(s) and/or sub-systems to which each of the received responses and/or questions in the questionnaire(s) pertains may be determined (step 220). A physiological system may be a macro grouping of a physiological system of a human being. Exemplary physiological systems include, but are not limited to, general health, ear, nose, & throat (ENT), cardiothoracic, gastrointestinal, circulatory, musculoskeletal, dermatologic, neurologic, mental health, hematological, and immune. Exemplary sub-systems include smaller systems and/or categories within a physiological system. A plurality of physiological symptoms/indicators may be associated with each physiological system and/or sub-system. In some instances, the physiological symptoms/indicators are specific to the associated physiological system. In some embodiments, the physiological systems determined in step 220 are determined by, for example, general requirements of, for example, a health insurance company, a federal agency (e.g., the Center for Medicare and Medicaid Services (CMS)), specific requirements of a physician's area of medical practice, a physician, medical facility, and so on. In some embodiments, the physiological system associated with execution of step 220 for a particular patient may be specific to the patient. In some embodiments, step 220 may be performed prior to step 215 so that, for example, received answers may be sorted into appropriate wellness score determinations.

Next, a statement regarding the patient's wellness with regard to one or more physiological systems, physiological sub-systems, and/or physiological symptoms associated with a particular physiological system may be prepared using, for example, the received responses (step 225). These statements may be referred to herein as “review of systems (ROS) statements.” Exemplary ROS statements include, but are not limited to, wellness scores, improvement scores, narrative statements, recommendations (e.g., follow up in 3 weeks, treatment recommendations, etc.), and/or severity evaluations (e.g., mild, moderate, severe). In some instances, the questions of the questionnaires and the responses thereto may be in non-medical language and preparation of the statement may condense, summarize, and/or translate the received responses into medical terminology. Exemplary ROS statements are provided by review of systems (ROS) window 438 of FIG. 4H, which provides a review of systems for a patient complaining of lack of energy, muscle aches, joint pain, and loss of motion. The review of systems window 438 provide a list physiological systems along with a corresponding narrative statement. In particular, the exemplary list of physiological systems provided by ROS window 438 includes general health, ear nose and throat (ENT), cardiothoracic, gastrointestinal, genitourinary, musculoskeletal, dermatologic, neurological, mental health, and heme/immune. Each of these physiological systems is associated with a corresponding statement pertaining to whether or not the patient is complaining of pay particular symptoms associated with the system. For example, the list indicates that the patient does not have any symptoms (e.g., no chest pain, racing heart, leg swelling, breathing, prolonged cough, wheezing, or coughing up blood) pertaining to his or her cardiothoracic physiological system.

At times, there may be a set of physiological systems for which statements may be prepared and, in step 230, it may be determined whether each physiological system of the set of physiological systems is associated with a prepared statement. If not, a statement saying that there are no indications that the patient is demonstrating a physiological symptom of a particular physiological system may be prepared (step 235). FIG. 4C provides a portion of medical note interface 400 that shows an exemplary set of physiological systems along with statements prepared via execution of step(s) 225, and/or 235.

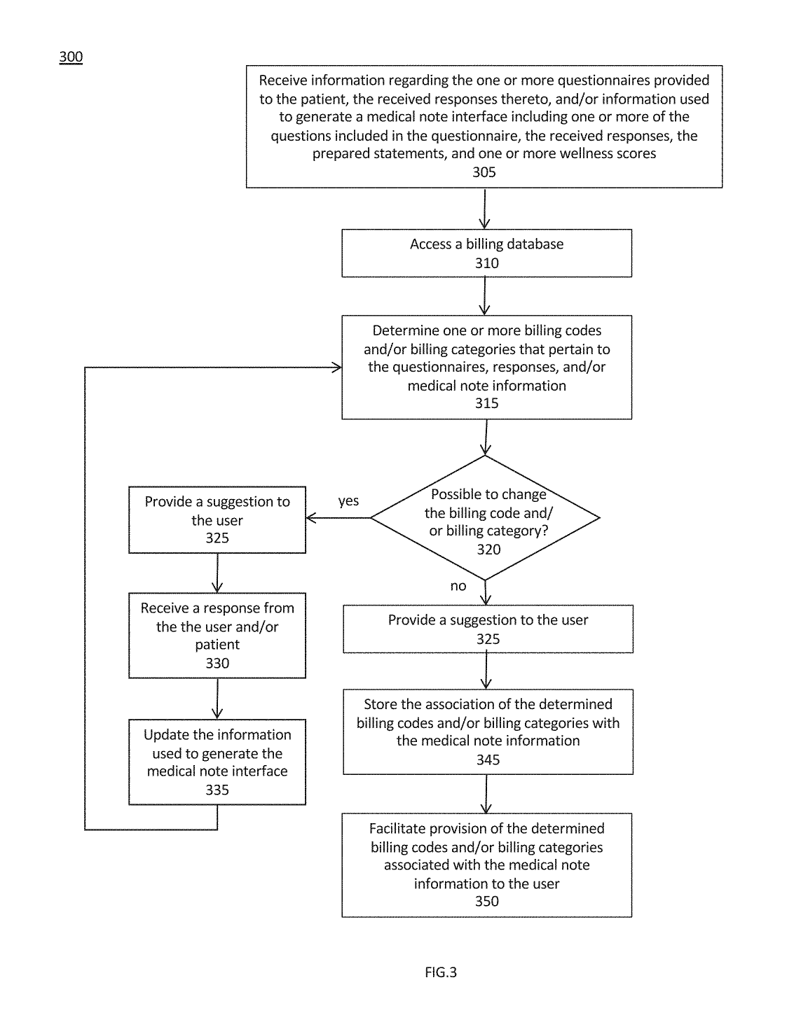

When there is a statement for each of the physiological systems to be associated with a medical note, it may be determined how the received responses, determined wellness score(s), and/or physiological system statements relate to one or more types of medical decision-making data (step 240). Medical decision-making data may be data that a physician uses to make a medical decision. The amount and complexity of the medical decision-making data may be useful in determining a billing code (e.g., and E&M code) for the care provided to the patient as will be described in greater detail below with regard to process 300 as illustrated in FIG. 3.

Next, a statement regarding the how the received responses relate to one or more types of medical decision-making data (step 245) may be prepared. In step 250, it may be determined whether a statement has been prepared for each type of medical decision-making data to be included on a medical note interface and, if not, a statement saying that there are no indications for an issue with a particular type of medical decision-making data may be prepared (step 255). Exemplary medical decision-making data may relate to data a physician uses to make a medical diagnosis or treatment decision. For example, in the medical note interface 408 of FIG. 4H, medical decision-making data is provided in the form of a wellness score for the patient's shoulder and each sub-medical questionnaire related to the patient's shoulder is provided along with changes in those scores (e.g., improvement or declination percentages) and wellness scores for the metabolic syndrome and diabetes medical questionnaires. Additionally, or alternatively, medical decision making data may include an initial review of systems, the patient's chief complaint, adverse life events and stresses, and information regarding physical function. Interface 408 may further provide an icon by which the user may enter a decision (e.g., a diagnosis, treatment decision, etc.) regarding, for example, medical decision-making data. For example, FIG. 4H provides a diagnoses category into which the clinician may enter one or more diagnoses for the patient via, for example, selection of the “+ add new diagnoses” icon, which may trigger display of an add diagnosis interface like add diagnosis interface 409 provided by FIG. 4I into which the user may enter, or search for, one or more diagnosis.

When a statement for each type of medical decision-making data is prepared, and/or following execution of step 255, one or more medical note interfaces like the medical note interfaces described herein may be prepared (step 160) and provision of the prepared medical note interface(s) may be facilitated (step 265) via, for example communication of the medical note interface(s) to a display device like display provided by user device 124 and/or patient device 128.

In some instances, execution of step(s) 225, 230, 235, 245, 250, 255, and/or 260 may include preparation of a narrative description (e.g., sentence, paragraph, or series of paragraphs) of the user's encounter with the patient and/or a summary of the patient's wellness, and/or medical condition that may be based on and/or include, for example, the received responses, prepared statements, template information, wellness scores, and/or improvement scores. The information populated into the narrative description may be drawn from, for example, the patient's EMR, the patient's wellness account, responses received in step 210, determined wellness scores, and/or improvement scores.

An example of a medical note interface that includes a narrative description is provided by medical note interface 412 of FIG. 4L. Medical note interface 412 provides a narrative paragraph near the top of the interface 412 that summarizes the patients basic health history (e.g., name, age, and gender), his chief complaint (“I can't walk more than a few blocks”), his medical status (“post cardiac resynchronization therapy 10/4/17”), medications he is currently taking (Furoserride 40 mg po bid), the patient's compliance with the medication (“8/10 compliance”) and a summary of the patient's wellness and/or improvement scores, which may relative to a previously received set of responses (in this case, responses received on 4/10, 3017). The narrative statement provided by interface 412 indicated the patient's shortness of breath has improved 12%, physical function has worsened 5%, anxiety has improved 5%, depression has improved 34%, and fatigue has worsened 5% which may be coded (e.g., bold and/or red for decline and underlined and/or green for improvement) to indicate, for example, improvement or decline and/or severity of an improvement or decline. The patient's compliance score pertains to how compliant the patient has been with taking the medication (Furoserride 40 mg po bid).

In some embodiments, the narrative description may employ one or more templates and/or user configured settings that pre-populate a text field or a portion thereof. For example, generation of a narrative description of a basic physical exam that gathers a patient's vital information may include a generic narrative description of the vital signs measured (e.g., blood pressure, weight, pulse) into which the observed values for a particular patient are populated. In some instances, a template may include text along with fillable fields in which to insert information specific to the patient and/or patient encounter. For example, a template for history of present illness (HPI) may be as follows (with patient-specific information shown in in brackets): [patient first name] [patient last name] ([xx] year old [male/female]) presents today with a chief complaint of “[CC]”. [He/She] was last seen by me on [date of last visit] and was most recently given a diagnosis of [diagnosis1] and [diagnosis2], and was prescribed [medication 1] and [medication 3]. [He/She] is most recently status post [procedure] on [procedure date] and [procedure] [procedure date]. [His/Her] most recent patient reported outcome questions and answers are as follows: . . . .

In another example, a template for the assessment/plan section of a medical note may be similar to the following (with patient-specific information shown in in brackets): [His/Her] patient reported outcome overall [overall CPT name] score today is [xx] out of a potential best of 100. This is a [interval % change] since [previous CPT score date], and a [overall % change] since [baseline CPT score date]. [He/She] reports the following adverse life events or stresses since [his/her] last visit: [adverse life event].

In some embodiments, only relevant portions (e.g., portions for which information is available) of a particular template may be provided as a part of the narrative included in a medical note. This way, a template narrative does not include language or text that is not needed and/or is unhelpful. For example, if a patient has no adverse life events reported in either their EMR or as a response to a medical questionnaire, then the sentence “[He/She] reports the following adverse life events or stresses since his last visit: [adverse life event]” may not appear in the template language/narrative discussion under the assessment/plan section of the medical note interface. Additionally, or alternatively, as statement saying, for example, “[He/She] reports the following adverse life events or stresses since his last visit: [none]” may appear in the template language/narrative discussion under the assessment/plan section of the medical note interface.

In some embodiments, one or more steps of process 200 may employ storage and/or communication of information using a decentralized and/or distributed ledger that, in some instances, may be maintained by an open peer-to-peer computer network and, in other instances, may be maintained by closed (e.g., a group of known computers operated by a particular entity) peer-to-peer computer network as, for example, a permissioned or private block chain. In many instances the distributed ledger will create and manage a block chain database.

In some embodiments, process 200, or portions thereof, may be executed before, during, or following a patient's visit or encounter with a physician or other professional (e.g., nurse, receptionist, billing specialist, nurse practitioner, etc.), which may be collectively referred to herein as a user. For example, step 205 may be executed prior to the patient's visit with a physician (e.g., when requesting an appointment with the physician or when checking in after arriving at the physician's office). Steps 210-260 may be at any point executed prior to the prior to the patient's visit with the physician and/or during the patient's visit with the physician. In many instances, step 265 may be executed just prior to the physician's visit with the patient (e.g., while the patient is in the waiting room) and/or while the patient is visiting the physician so that the physician may, for example, review the medical note for accuracy, ask follow-up questions, request diagnostic tests, and so on while the physician has physical access to the patient. Additionally, or alternatively, the physician may review the medical note after the patient has left the physician's office (e.g., at the end of the day). In some instances, review of the medical note may include entry of the physician's impressions of the patient's wellness and/or medical condition into the note via, for example, a text entry field.

FIGS. 4J-4L provide other exemplary medical note interface(s) 410, 411, and 412 respectively, that may be prepared via execution of one or more steps of process 200. The information displayed on medical note interface 410 is separated into a few categories which are shown as windows (or areas of the interface). For example, medical note interface 410 includes a chief complaint window 440, a history of present illness (HPI) window 442, review of systems window 438, a medical history and assessment window 446, and a medical decision-making data window 445. Chief complaint window 440 may include a narrative description of the patient's chief medical complaint. This information may be a direct quotation from the patients regarding their chief complaint and/or the user's and/or a caregiver's assessment of the patient's chief complaint. HPI window 442 may, for example, include a listing of one or more medical questionnaires provided to the patient, one or more answers received from the patient, one or more questions provided to the patient, and/or one on more determinations or evaluations of the patient's wellness (e.g., wellness score) and/or improvement (e.g., improvement score). Review of symptoms window 438 may include a list of, for example, positive symptoms applicable to the patient and/or a list of one or more symptoms that are not applicable to the patient. Medical history and assessment window 446 may include a variety of information regarding the patient's medical history, examinations performed, personal history, demographics, and/or lifestyle. In the example of interface 410, medical history and assessment window 446 includes past family and socially history information, including adverse life events that may have an impact on the patient's health, a place to enter information regarding physical exams of the patient, and a place to enter information regarding assessments and plans for the patient. Medical decision-making data window 445 may include variety of information that the user may use to make medical decisions including, but not limited to, wellness scores and improving scores that have been determined responsively to answers received to one or more medical questionnaires.

Medical note interface 410 may include one or more text boxes 444 or other fillable form areas in which a user may enter (via, for example, typing, icon selection, and/or dictating information, which may be translated into text) notes or observations into the medical note for the patient. This allows the user to enter free-form descriptions of, for example, the patient's general state of health, mood, degree of mobility, medical condition, and so on. At times, the information entered may not relate to a particular question previously provided to the patient via a medical questionnaire and may be an answer to a question the doctor asks the patient when they are face-to-face. In some instances, it may be a general impression of a patient that may be difficult to capture via the process of the patient's answering medical questions. In some instances, these notes may be entered for specific categories of information, but this need not always be the case. Medical note interface 410 provides an example of an interface that includes text entry boxes positioned under the category headings of history of present illness (HPI), review of systems (ROS), past family and social history (PFSH), physical exam (EXAM), assessment & plan (AP), and medical decision making data. Medical note interface 411 of FIG. 4K provides a medical note interface similar to medical note interface 410 except that a note (“Patient complained of additional pain shooting down spine”) has been added to the HPI category.

Medical note interface may include one or more graphic elements 448 that enable the user to indicate that he or she has reviewed and/or approved of the information populated into medical interface 410. For example, a user may sign the medical note interface indicating his or her review and approval of the information included thereon by selecting graphic element 448. In some instances, selection of graphic element 448 may trigger the display of another interface by which the user may enter information that is personally identifiable (e.g., password, username, and/or bioinformatic information) so as to securely sign or otherwise indicate approval of the information included in the medical note interface 410.

In the example of medical note interface 400, this indication may be received when the physician selects the “review and save” icon shown in FIG. 4F. In the example of medical note interface 410, this indication may be received when the physician selects the “sign” icon shown in FIG. 4J. When an indication that the user (e.g., physician or other medical care provider) has reviewed and approved the information provided by the medical note interface is received (step 270). Optionally, a medical note interface may then be locked (step 275) so that it may not be further modified. In some instances, there may be a graphic indication that the information on the medical note interface has been reviewed and approved (e.g., the text “reviewed” may be displayed on the interface). In addition, the text “pending review” may change to “reviewed.” Additionally, or alternatively, an indication of a status of the medical note under review may change to signed/reviewed/approved upon receipt of the indication. For example, medical note interface 410 show that the medical note interface is unsigned and medical note interface 411 of FIG. 4K indicates that the note is signed by Dr. Patel on Oct. 10, 2017. In addition, the “sign” icon of medical note interface 411 has changed to an “addend” icon responsively to receipt of the indication. Selection of the addend icon may enable the user to revise and/or edit the entries to the text boxes. These revisions/edits may then be saved.

Then, the locked medical note interface may be stored (step 280) and, in some instances, a notification, such as a message, icon, or color change may be provided by the medical note interface to indicate that the medical note has been locked and/or saved. In some embodiments, the locking and/or storing of steps 275 and 280 may include packaging a medical note interface and/or information associated therewith for storage as a data object stored on a block chain.

Optionally, the locked medical note may be associated with the patient's EMR and/or personal medical/patient account (step 285). The user's selection of “reviewed” and/or the locking of the medical note interface may serve to document that the user has reviewed the content of the note. This may be helpful when documenting services provided, time spent with the patient, and/or information considered by the clinician when treating the patient for accounting and/or billing purposes.

Steps 275-285 may be executed at any point following execution of step 270. Often times, steps 275-280 are executed immediately following execution of step 270.

FIG. 3 provides a flowchart illustrating an exemplary process 300 for determining a billing code of a patient's visit to a user (e.g., doctor, clinician, etc.). Process 300 may be executed by, for example, a server in communication (via, e.g., the Internet or a secure network) with, for example, a patient's electronic device, an electronic device operated by a medical facility and/or medical facility computer system. Process 300 may be executed by, for example, systems 100, 150, and/or 1100 and/or portions thereof.

Initially, information used to generate a medical note interface, like one or more of the medical note interface(s) generated via execution of process 200, may be received (step 305). More specifically, information regarding the one or more questionnaires provided to the patient, the received responses thereto, and/or information used to generate a medical note interface including one or more of the questions included in the questionnaire, the received responses, the prepared statements, wellness scores, and/or one or more wellness scores via execution of, for example, process 200 or a portion thereof may be received in step 305. Then, a billing database, such as billing database 180 may be accessed (step 310) using, for example, billing program 138 in order to determine one or more billing codes and/or billing categories that pertain to the information received in step 305 (step 315).

In step 320 it may be determined whether it is possible to change the billing code and/or billing category by, for example, performing a diagnostic test or asking a series of follow up questions relating to, for example, a medical condition, treatment side effect, chief complaint, and/or co-morbidity of the patient. In some cases, the change in the billing code/billing category may be have an effect of upgrading the billing code/billing category because more information and/or more medical decision-making data may be received, available, and/or reviewed by the user.

When there are possible ways to change the billing code/billing category, a suggestion for how to effect such a change may be determined and/or provided to the user (step 325). A response to the suggestion (e.g. accepting or declining the suggestion in whole or in part) may then be received (step 330) and information used to generate the medical note interface may then be updated (step 335). Process 300 may then proceed to step 310.

In some cases, one or more of steps 305-235 may be performed prior to completion of step 270 so that a medical note interface provided to the user may be updated to include the response to the suggestion. When this is not possible, an appendix or other supplemental information may be appended to a medical note interface, and in some instances, a locked medical note interface.

When there are no possible changes to the medical note interface determined in step 320 and/or when the user does not want to provide a response to the suggestion, the determined the billing code/billing category may be associated with the medical note interface and/or medical note information (step 345). Then, provision of the determined the billing code/billing category associated with the medical note interface and/or medical note information (step 350) may be facilitated.

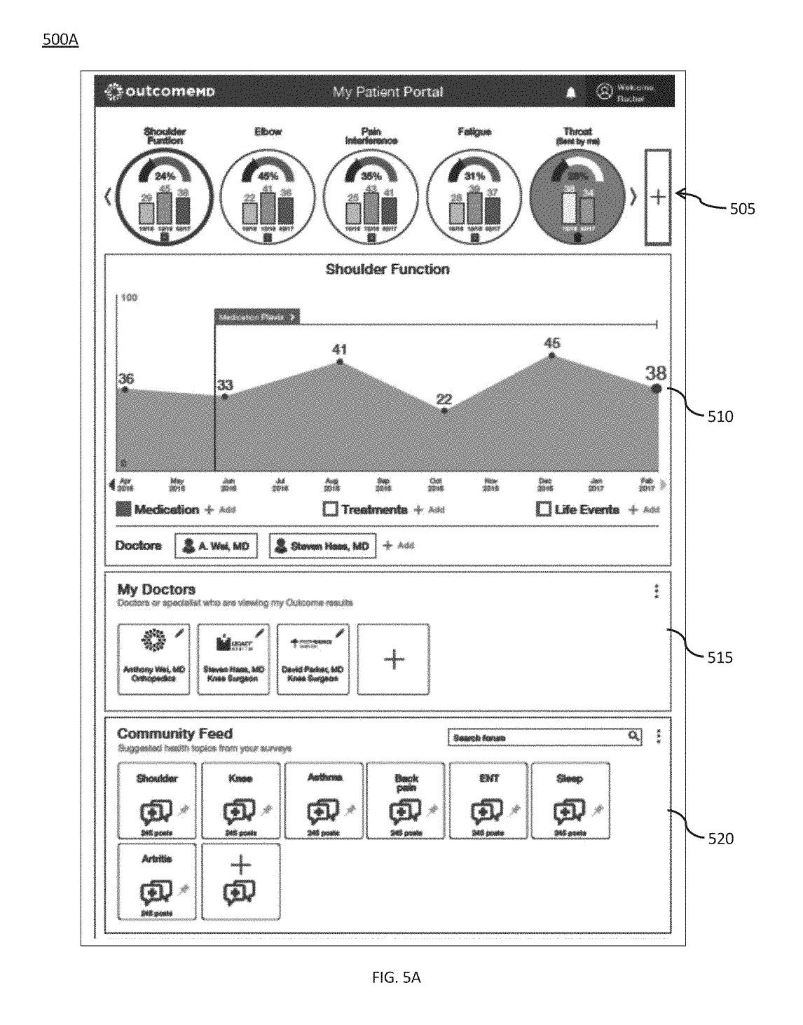

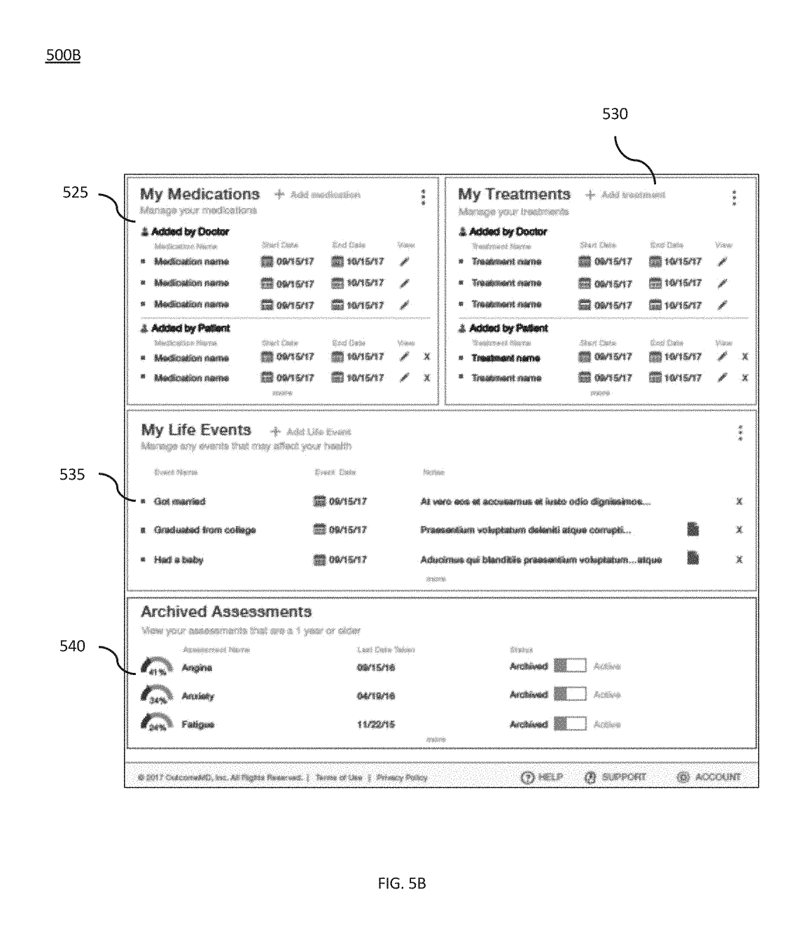

FIGS. 5A-10 provide a number of exemplary interfaces by which a user (e.g., physician, clinician, etc.) patient, and/or caregiver for the patient may interact with information associated with a medical note interface, a patient's wellness account and/or EMR. A patient wellness account may be any set of information associated with the patient and his or her wellness. It may be maintained and/or populated with information from the patient, a user, and/or a software application providing the patient account. The software application providing the patient account may be available to the patient on demand via his or her electronic device and, often times, will require compliance with a security measure (e.g., entry of a valid password or biometric data (e.g., fingerprint)). In some embodiments, a patient may be able to add information to one or more medical note interfaces described herein via his or her patient account. For example, the patient may receive the medical questionnaires provided by step 205 via software running on an electronic device (e.g., smart phone or tablet computer) his or her patient account that presents the questions of the medical questionnaire to the patient. The patient may also access and/or view information like that provided in the screen shots of FIGS. 5-10.

FIGS. 5A and 5B provide first and second portions of an exemplary overview dashboard interface 500A and 500B, respectively for a patient account that may be provided to a patient and/or caregiver (which may be collectively referred to herein as “patient”) upon, for example, accessing the software application that provides the patient account. Dashboard interface 500A includes a wellness score/system/symptom type selection window 505, a wellness score window 510, a my doctors window 515, a community feed window 520, and dashboard interface 500B includes a my medications window 525, a my treatments window 530, a my life events window 535, and an archived assessments window 540.

A patient may access information regarding one or more diagnoses, wellness scores, physiological systems, and/or physiological symptom types via interaction with wellness score/system/symptom type selection window 505. Exemplary information the patient may access via interaction with window 505 includes, but is not limited to, wellness scores, treatments, medications, life events, doctors, and improvement scores that may be related to a diagnoses, physiological system, and/or physiological symptom for the patient provided by window 505. For example, window 505 indicates that the patient has a shoulder wellness score of 34%, an elbow wellness score of 45%, a pain interference wellness score of 35%, a fatigue wellness score of 31%, and a throat wellness score of 38%. Each of these wellness scores may have been determined via execution of any of the processes disclosed herein.

In the embodiment of FIG. 5, the shoulder function, elbow, pain interference, and fatigue wellness scores were determined responsively to medical questionnaires pertaining to these physical systems being sent to the patient by a user. The throat wellness score was determined responsively to a medical questionnaire the patient sent him- or herself as indicated by the “(sent by me)” parenthetical.

Wellness score window 510 may provide one or more wellness scores for a diagnosis, physiological system, and/or physiological symptom type. At times, the wellness score information may be displayed responsively to a selection from wellness score/system/symptom type selection window 505. In the example of interface 500, the wellness score information displayed in wellness score window 510 relates to shoulder function and may be displayed responsively to a patient selecting the left-most icon provided by wellness score/system/symptom type selection window 505 pertaining to shoulder function. The wellness score information is displayed in wellness score window 510 as a graph showing wellness scores for shoulder function (on the Y-axis) determined over time (on the X-axis). A patient may scroll to the left or right to access wellness scores for shoulder function determined earlier and/or later in time.

In some embodiments, a patient may add information (other than wellness scores) to the information displayed in window 510 by selecting one or more icons provided by window 510. For example, window 510 provides icons that facilitate the addition of medication, treatments, and life events to the information displayed on the graph of window 510. In some cases, the medication, treatments, and/or life events may have specific relevance to the medical condition (in this case, shoulder function) selected by the patient and, in other cases, they may not (e.g., a medication taken by the patient that is not related to shoulder function may be displayed on the graph of window 510).

The medication, treatment, and/or life event may also be correlated in time to the wellness scores displayed in window 510. For example, in the graph of display window 510, a patient has requested information relating to medication be displayed (see grey box next to medication) and a graphic element in the form of a vertical line between May and June of 3016 indicates when treatment with the medication began and the horizontal line extending from the vertical line indicates how long the patient has been taking the medication. The rectangle above the vertical line indicates the name of the medication, in this instance, the medication is Plavix.

Window 510 also provides a list of doctors/clinicians that have access to and/or are in some way associated with the patient's shoulder function information. A patient may add users, doctors, and/or clinicians to this list by selecting the add icon.

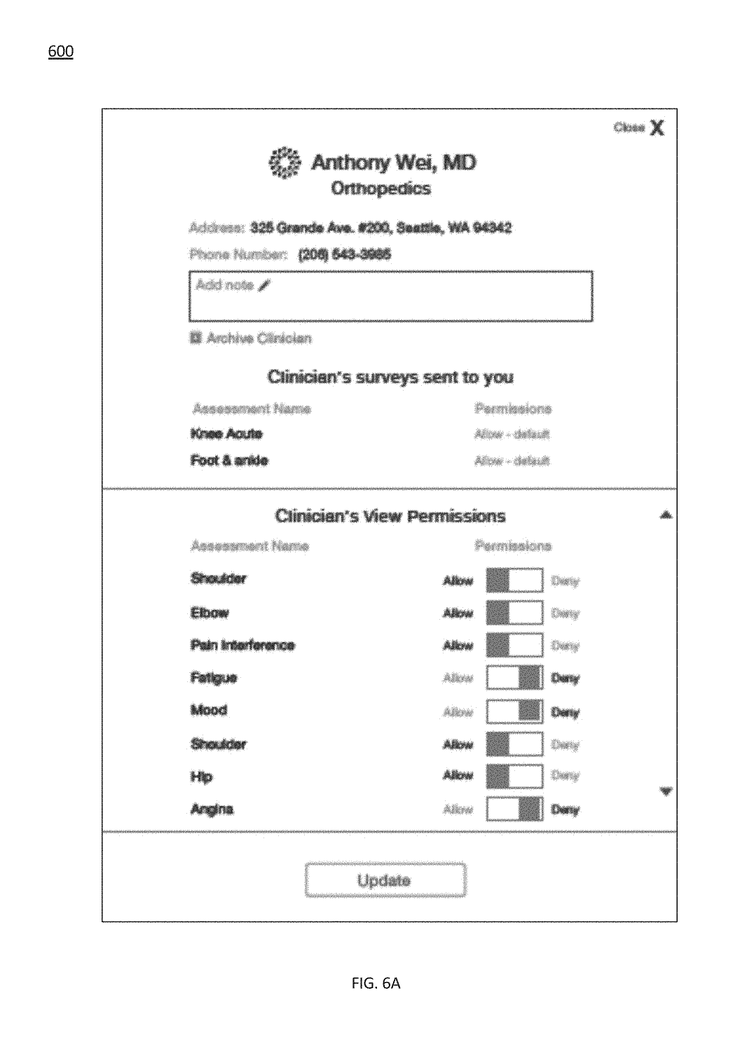

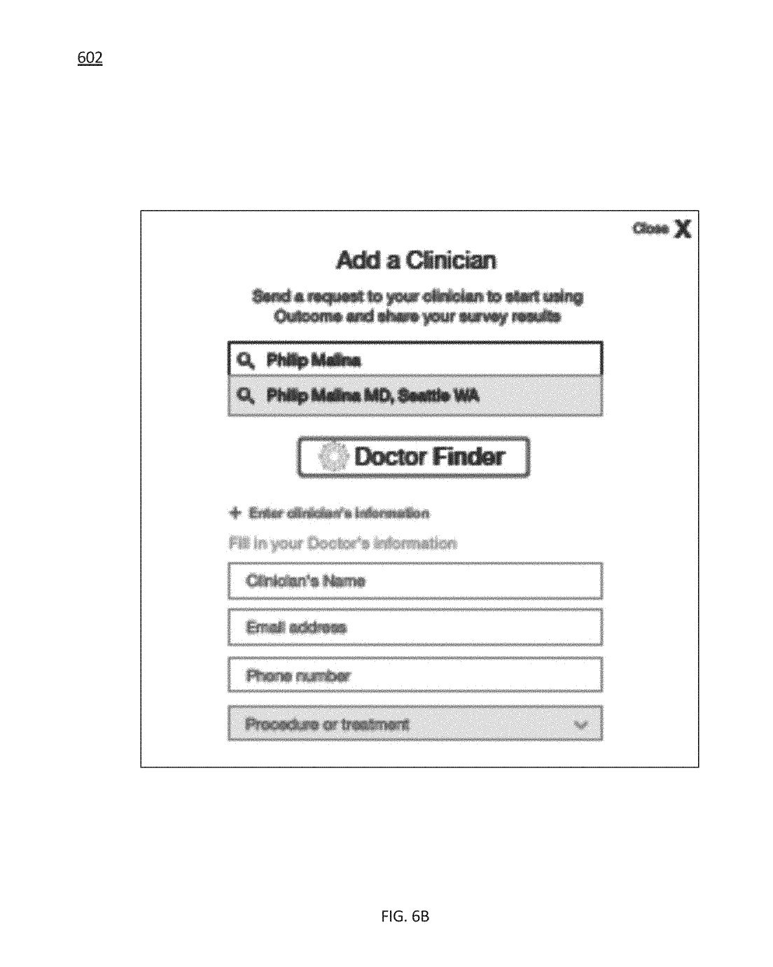

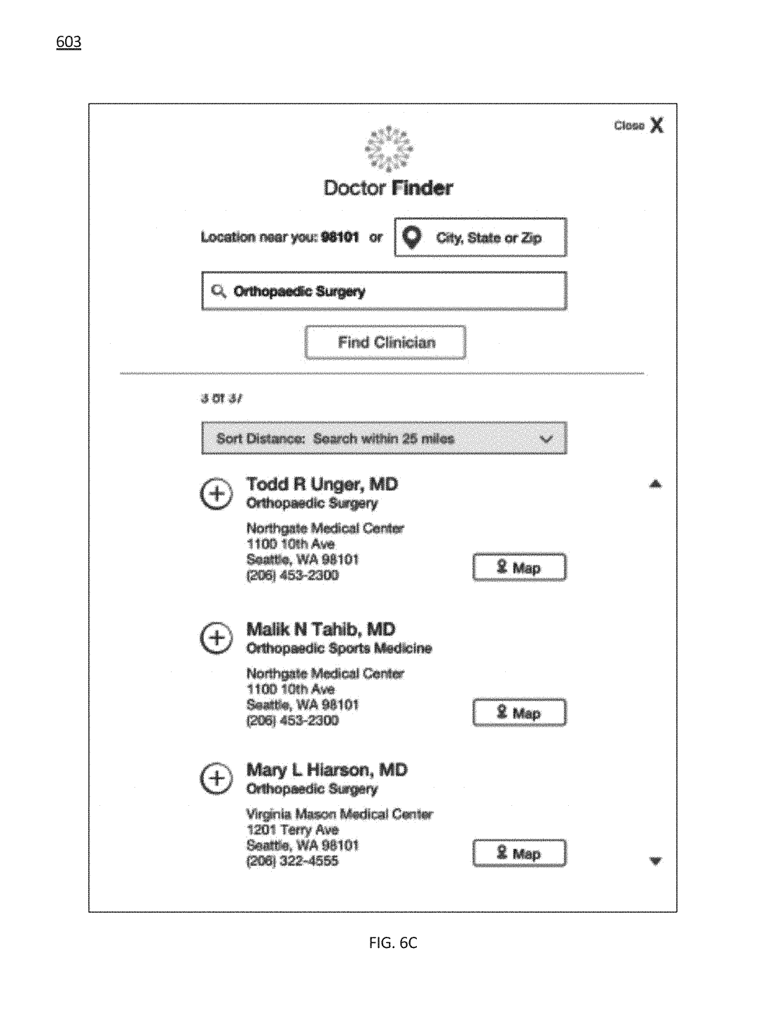

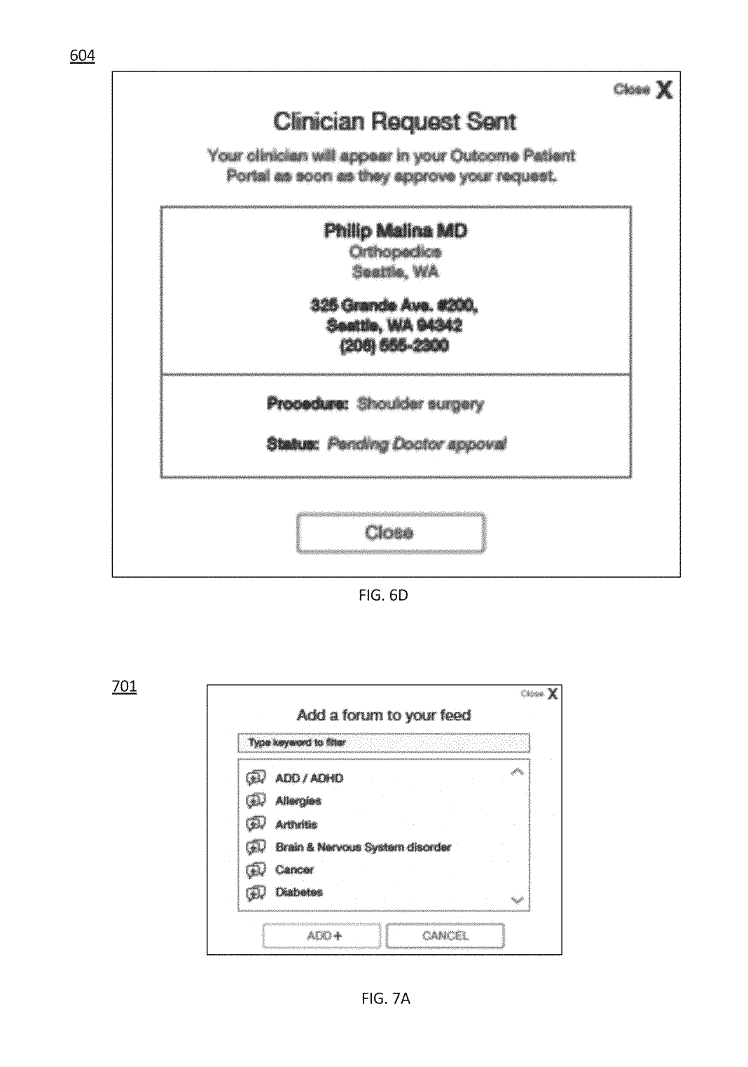

My doctors window 515 displays an array of icons representing three different doctors who are associated with and/or have viewing privileges for the patient's wellness account and/or a diagnosis, treatment, or symptom. Each doctor's icon may include identifying information (e.g., name, specialty, medical group/hospital, and/or location). Upon selection of a particular doctor's icon (in this case, Anthony Wei, MD), a window, such as exemplary clinician's view permissions interface 601 as shown in FIG. 6A, may be provided to the user. Clinician's view permissions interface 601 provides information regarding, for example, the name, address, and specialty of the clinician, which medical questionnaires (referred to as “clinician's surveys sent to you”) have been sent to the patient, and an interface that facilitates the patient's selection of which types of information the doctor has permission to see.

A patient may add one or more users and/or doctors to the my doctors array via selection of the “+” icon provided by window 515 of interface 500. Upon selection of this icon, a window, such as exemplary add a clinician interface 602 as shown in FIG. 6B may be displayed. A patient may enter a clinician name into the text box provided by interface 602 and it may auto-populate with one or more known clinicians. Additionally, or alternatively, a patient may enter the clinician's name, address, etc. into text boxes provided by interface 602. Additionally, or alternatively, a patient may search for a clinician via selection of the doctor finder icon, which may facilitate display of a doctor finder window such as doctor finder interface 603, which is provided by FIG. 6C.

Use of doctor finder interface 603 may facilitate the patient's searching for a doctor and/or clinician. In the upper portion of doctor finder interface 603, a patient may enter a location (e.g., city, state, or zip code) into a first text box and a medical specialty into a second text box, which may also be implemented as, for example, a drop down menu. A list of clinicians that meet one or more criterion (e.g., specialty, location, gender, etc.) may be displayed following, for example, entry of the location and medical specialty and/or upon selection of the find clinician button. The list of clinicians provided by interface 603 may include any number of clinicians that meet the entered criteria. When a clinician is selected (via, for example, selection of the “+” icon on interface 603 adjacent to the selected clinician), a clinician request interface such as clinician request interface 604 may be provided to a display device. Via interface 604, the patient may send and/or receive a request to communicate with the selected clinician so as to, for example, request information and/or scheduling of a treatment (e.g., shoulder surgery, wellness checkup, etc.).

The next window provided by interface 500 is the community feed window 520 by which a patient may gain and/or manage access to one or more subject areas, or forums, for the patient's feed of information. The patient's community feed may be a platform for the sharing of information between various patients, clinicians, and/or caregivers who may be associated with one or more physiological systems, physiological symptoms, diagnoses, treatments, and so on. Community feed window 520 provides a number of icons, each of which represent a different forum, or subject area, that the user has subscribed to. In the example of community feed window 520, the exemplary forms shown include a form for shoulder, knee, asthma, back pain, ENT (ear, nose, throat), sleep, and arthritis. The icons also provide a number of posts to the particular forum. For example, the shoulder icon 345 posts. The number of posts for a particular forum may assist the patient with assessing the richness information associated with the forum.

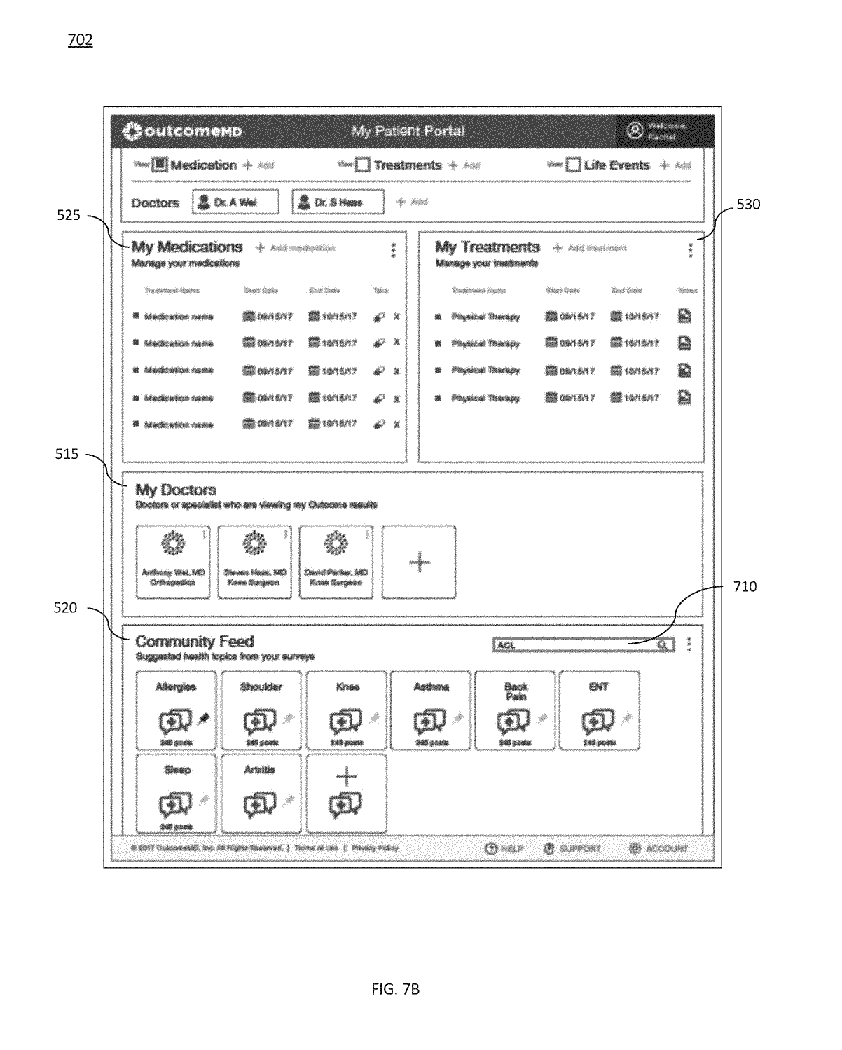

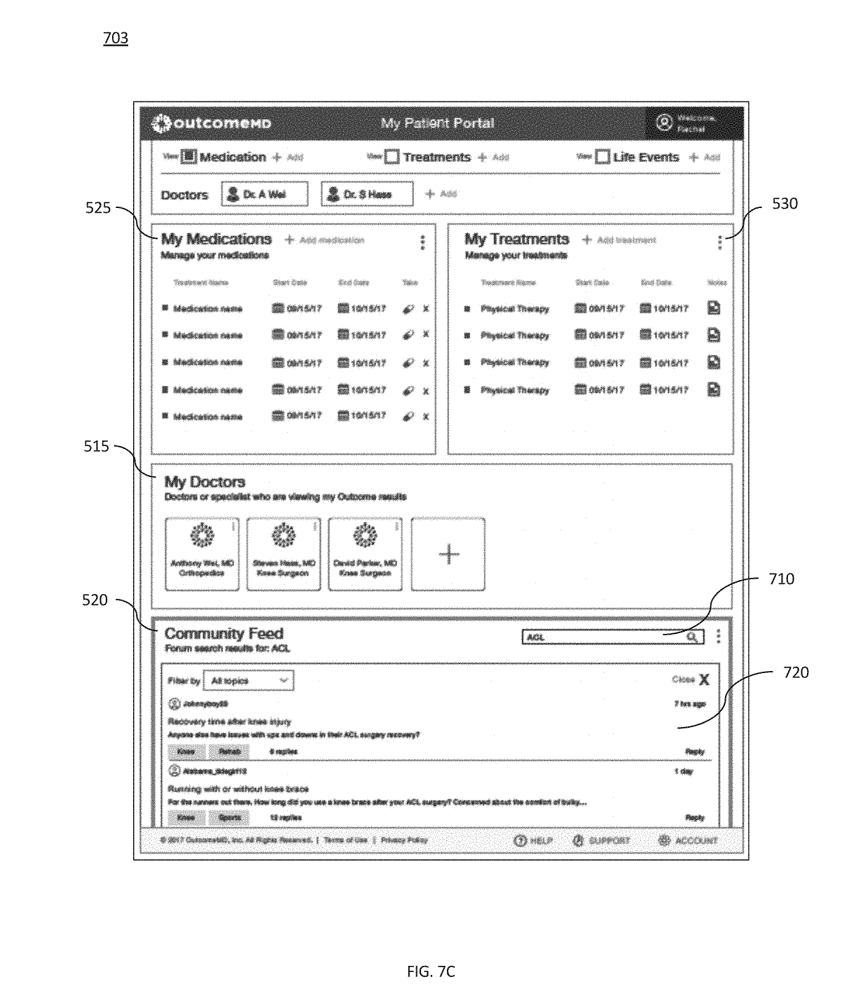

A patient may add a forum to his or her community feed via selection of the add (“+”) icon. When this icon is selected, an add a forum to your feed interface 701, as shown in FIG. 7A, may be displayed to the patient. The add a forum to your feed interface 701 may provide a text box by which the patient may enter one or more keywords that may be used to search for an associated forum and a list of available forums that may be scrolled through via, for example, up and down arrows or gestures. A selected forum may be added to the patient's community feed upon selection of and add icon as provided by interface 701.

Additionally, or alternatively, a patient may search for and/or add a forum to his or her community feed via entry of text into a search text box provided by community feed window 520 as shown in interface 702 of FIG. 7B. In the example of FIG. 7B, the patient has entered “ACL” into a text box 710 of community feed window 520. Following this entry, exemplary search research results for forums that mention “ACL” are provided in a list as shown in community feed interface 703 of FIG. 7C. The search results displayed in a community feed display window of community feed interface 703 and provide a series of comments, suggestions, and links to information that are relevant to the search for ACL. Each of the entries in the community feed display window is also associated with one or more categories (in this case, the first entry is associated with the categories of knee and rehab and the second entry is associated with the categories of knee and sports).

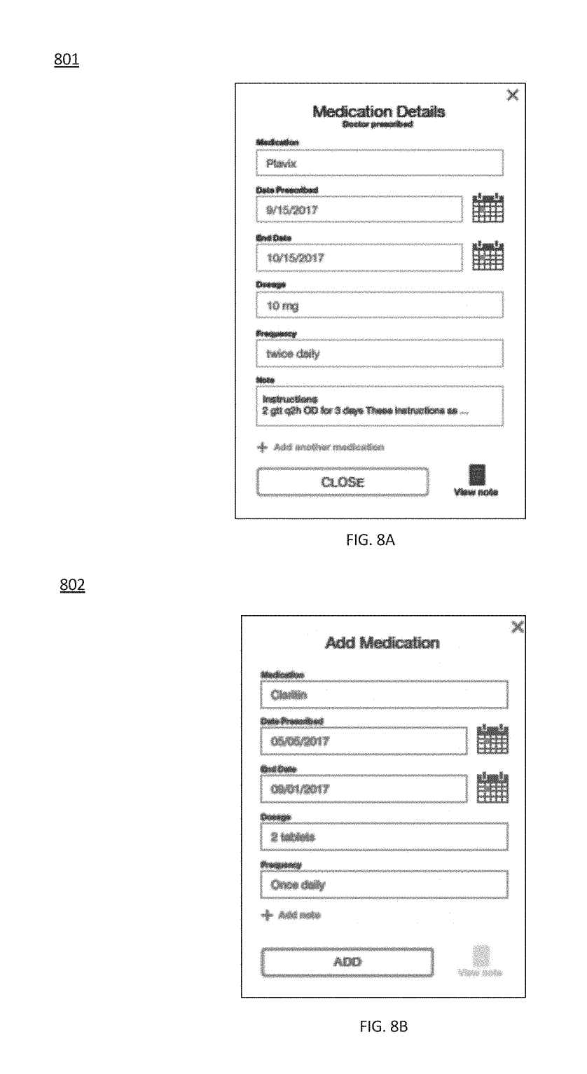

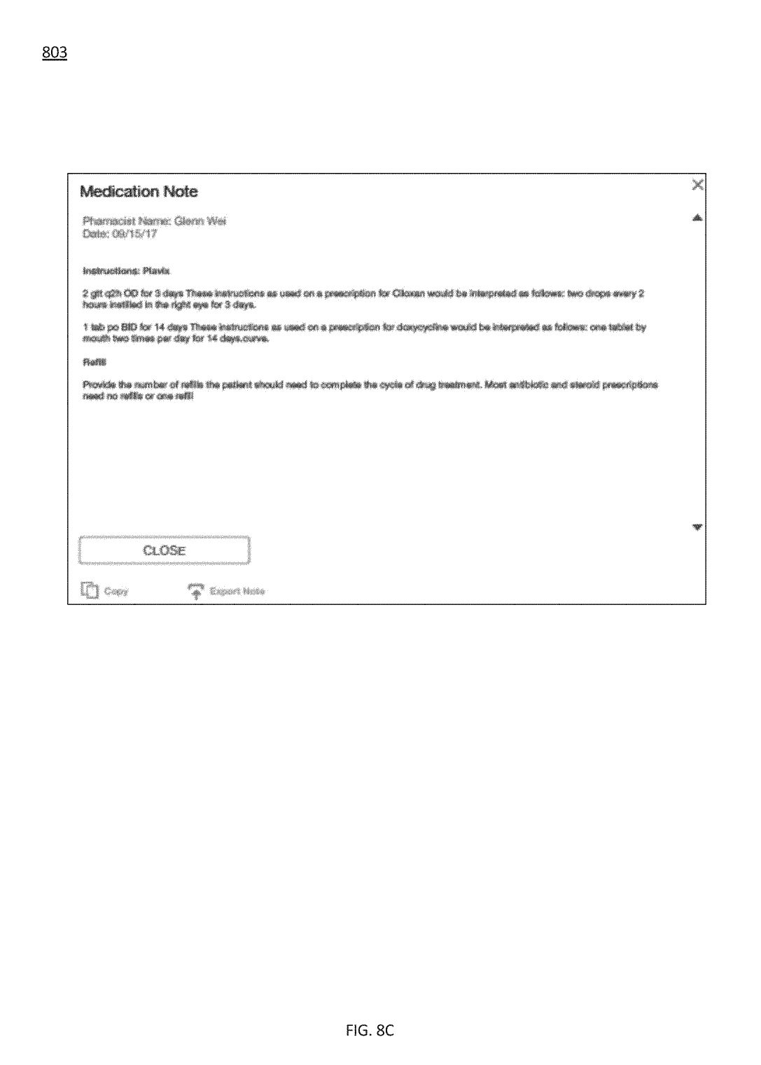

My medications window 525 may provide a display of a list of a number of medications the patient is taking and/or has been prescribed to take along with medication name, dosage amount, and when prescribed. In the embodiment of FIG. 5, the medications fall into two categories: added by doctor and added by patient. The medications added by a doctor may be added to the patient's account by his or her clinician and/or the patient. A patient may add a medication prescribed by a doctor/clinician to his or her patient's account via, for example, a medication details interface 801, an example of which is provided in FIG. 8A. Medication details interface 801 provides a number of text and information entry options for the entry of the medication name, start and end dates for taking the medication, dosage information, frequency information, and/or notes, which may include special instructions or other information.

A patient may add a medication that may not be prescribed by a doctor via, for example, selection of the “+ add medication” icon provided in my medications window 525. Upon selection of this icon, an add medication interface 802, as shown in FIG. 8B, may be displayed. Add medication interface 802 provides a number of text and information entry options for the entry of the medication name, start and end dates for taking the medication, dosage information, frequency information, and/or notes, which may include special instructions or other information. The add medication interface 802 may be used by the patient to enter self-prescribed medications and/or treatments to the patient account. Exemplary self-prescribed medications include, but are not limited to, dietary changes, vitamins, supplements, etc.

Following entry of information into add medication interface(s) 801 and/or 802, a medication note, like the medication note provided by medication note interface 803 of FIG. 8C may be provided to the user. Medication note interface 803 may include an articulation of medication information as shown in FIG. 8C.

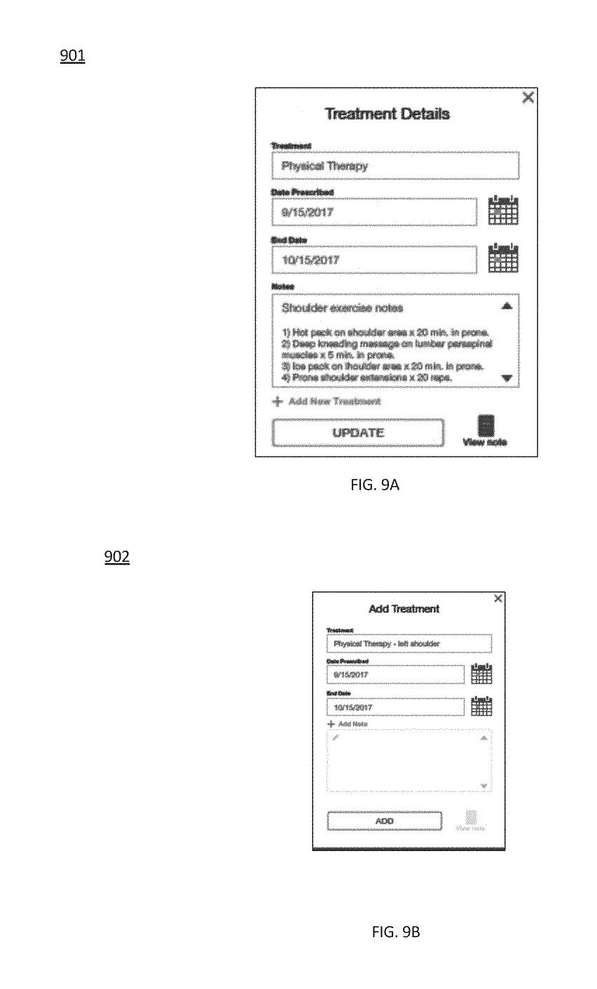

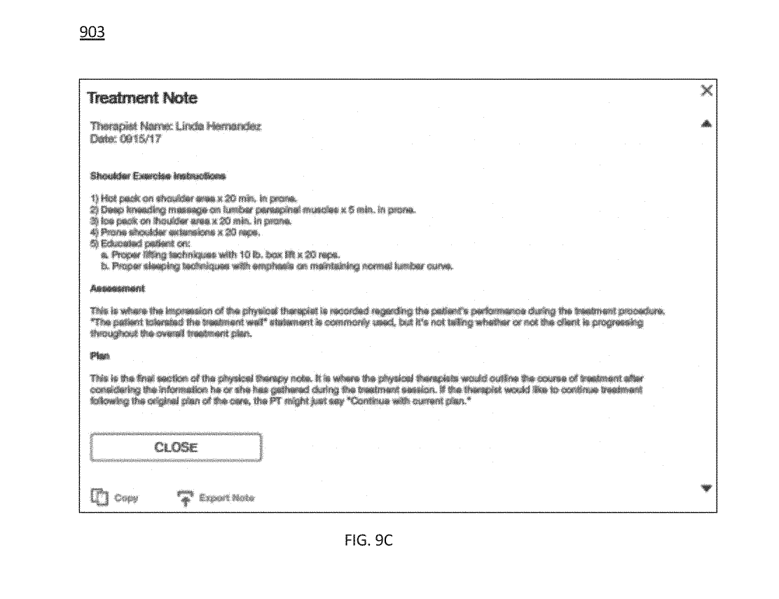

My treatments window 530 may provide a display of a list of a number of treatments the patient has undergone, is undergoing, and/or is scheduled to undergo that includes treatment name and a date range for the treatment. In the embodiment of FIG. 5, the treatments fall into two categories: added by doctor and added by patient. The treatments added by a doctor may be added to the patient's account by his or her clinician and/or the patient. A patient may add a treatment prescribed by a doctor/clinician to his or her patient's account via, for example, a treatment details interface 901, an example of which is provided in FIG. 9A. Treatment details interface 901 provides a number of text and information entry options for the entry of the treatment name, start and end dates for undergoing the treatment, and/or notes, which may include special instructions or other information.

A patient may add a treatment that may not be prescribed by a doctor via, for example, selection of the “+ add treatment” icon provided in my treatments window 530. Upon selection of this icon, an add treatment interface 902, as shown in FIG. 9B, may be displayed. Add treatment interface 902 provides a number of text and information entry options for the entry of the treatment name, start and end dates for taking the treatment, dosage information, frequency information, and/or notes, which may include special instructions or other information. The add treatment interface 902 may be used by the patient to enter self-prescribed treatments and/or treatments to the patient account. Exemplary self-prescribed treatments include, but are not limited to, dietary changes, exercise routines, stress management exercises, etc.

Following entry of information into add treatment interface(s) 901 and/or 902, a treatment note, like the treatment note provided by treatment note interface 903 of FIG. 9C may be provided to the user. Treatment note interface 903 may include an articulation of treatment information as shown in FIG. 9C.

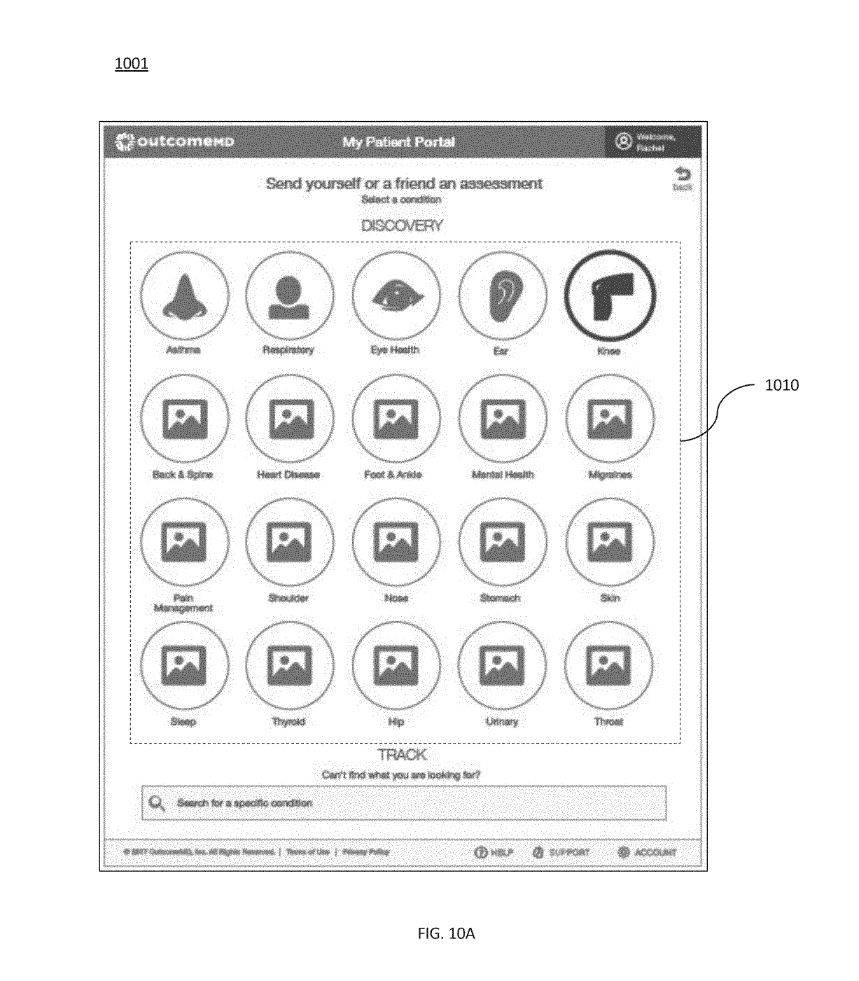

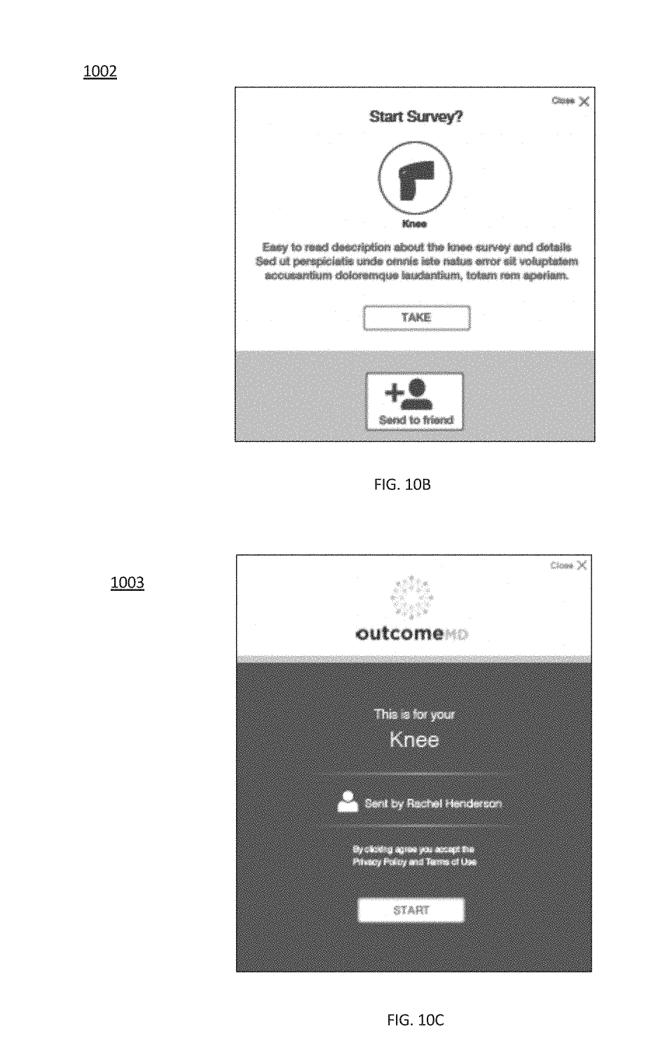

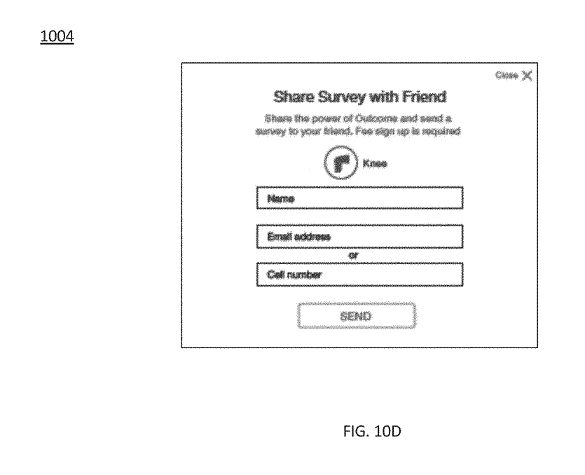

FIG. 10A provides an exemplary medical questionnaire (referred to on the interface as assessment) request interface 1000. Medical questionnaire request interface 1000 provides an array of icons 1010, each of which correspond to a medical condition, physiological system, physiological symptom, or other health-related concern. A patient may request one or more medical questionnaire to be sent to himself/herself or another individual (e.g., friend, family member, co-worker, etc.) via selection of one or more of the icons provided by medical questionnaire request interface 1000. A patient may also search for a medical questionnaire related to a particular topic by entering information related to the topic into a searching text box provided by the interface. For example, if a patient selects the icon of array 1010 corresponding to a knee medical questionnaire, a start medical questionnaire (referred to as a survey on interface 1002) interface 1002, indicating that the medical questionnaire to be started regards the knee as shown in FIG. 10B may be provided. The patient may then select a graphic element (e.g., a “take” icon) provided by interface 1002 so that he or she may answer the questions directly and/or select a “send to a friend” icon so that he or she may communicate the knee medical questionnaire to his or her friend or associate.

When the patient selects the “take” icon, a medical questionnaire introduction interface 1003 as shown in FIG. 10C may be provided to the patient so that he or she may commence with answering the questions and providing requested information for the knee medical questionnaire upon selection of the start icon.

When the patient selects the “send to a friend” icon, a “share the survey with friend” interface 1004 as shown in FIG. 10D may be provided to the patient so that he or she may enter information (e.g., name, email address, and/or mobile phone/cell number to facilitate communication of the knee medical questionnaire to the patient's friend. Then the patient may select send, which may facilitate communication of the knee medical questionnaire to the email and/or mobile phone number of the patient's friend as, for example, a text or SMS message.

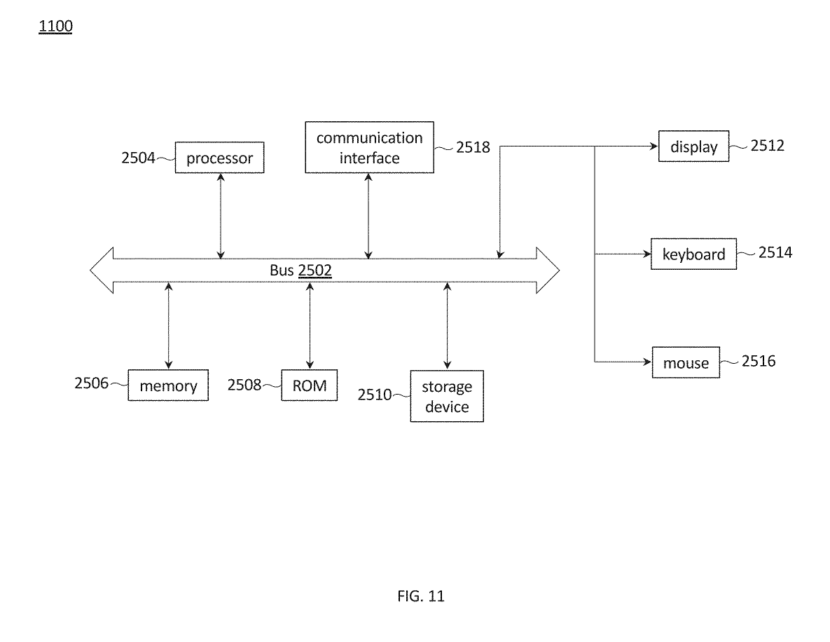

FIG. 11 depicts components of a computer system 1100 in which computer readable instructions instantiating the methods of the present invention may be stored and executed. As is apparent from the foregoing discussion, aspects of the present invention involve the use of various computer systems and computer readable storage media having computer-readable instructions stored thereon. FIG. 11 provides an example of a system 1100 that may be representative of any of the computing systems (e.g., server 102, treatment provider device 124, patient device 128, user device 124, treatment facility computer system 134, etc.) discussed herein. Examples of system 1100 may include a smartphone, a desktop, a laptop, a mainframe computer, an embedded system, etc. Note, not all of the various computer systems have all of the features of system 1100. For example, certain ones of the computer systems discussed above may not include a display inasmuch as the display function may be provided by a client computer communicatively coupled to the computer system or a display function may be unnecessary. Such details are not critical to the present invention.

System 1100 includes a bus 1102 or other communication mechanism for communicating information, and a processor 1104 coupled with the bus 1102 for processing information. Computer system 1100 also includes a main memory 1106, such as a random access memory (RAM) or other dynamic storage device, coupled to the bus 1102 for storing information and instructions to be executed by processor 1104. Main memory 1106 also may be used for storing temporary variables or other intermediate information during execution of instructions to be executed by processor 1104. Computer system 1100 further includes a read only memory (ROM) 1108 or other static storage device coupled to the bus 1102 for storing static information and instructions for the processor 1104. A storage device 1110, for example a hard disk, flash memory-based storage medium, or other storage medium from which processor 1104 can read, is provided and coupled to the bus 1102 for storing information and instructions (e.g., operating systems, applications programs and the like).

Computer system 1100 may be coupled via the bus 1102 to a display 1112, such as a flat panel display, for displaying information to a computer user. An input device 1114, such as a keyboard including alphanumeric and other keys, may be coupled to the bus 1102 for communicating information and command selections to the processor 1104. Another type of user input device is cursor control device 1116, such as a mouse, a track pad, or similar input device for communicating direction information and command selections to processor 1104 and for controlling cursor movement on the display 1112. Other user interface devices, such as microphones, speakers, etc. are not shown in detail but may be involved with the receipt of user input and/or presentation of output.

The processes referred to herein may be implemented by processor 1104 executing appropriate sequences of computer-readable instructions contained in main memory 1106. Such instructions may be read into main memory 1106 from another computer-readable medium, such as storage device 1110, and execution of the sequences of instructions contained in the main memory 1106 causes the processor 1104 to perform the associated actions. In alternative embodiments, hard-wired circuitry or firmware-controlled processing units may be used in place of or in combination with processor 1104 and its associated computer software instructions to implement the invention. The computer-readable instructions may be rendered in any computer language.

In general, all of the above process descriptions are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as computer system 1100 or similar electronic computing device, that manipulates and transforms data represented as physical (electronic) quantities within its registers and memories into other data similarly represented as physical quantities within its memories or registers or other such information storage, transmission or display devices.

Computer system 1100 also includes a communication interface 1118 coupled to the bus 1102. Communication interface 1118 may provide a two-way data communication channel with a computer network, which provides connectivity to and among the various computer systems discussed above. For example, communication interface 1118 may be a local area network (LAN) card to provide a data communication connection to a compatible LAN, which itself is communicatively coupled to the Internet through one or more Internet service provider networks. The precise details of such communication paths are not critical to the present invention. What is important is that computer system 1100 can send and receive messages and data through the communication interface 1118 and in that way communicate with hosts accessible via the Internet. It is noted that the components of system 1100 may be located in a single device or located in a plurality of physically and/or geographically distributed devices.

1. A method comprising:
receiving, by a processor, responses to a plurality of questions included in a medical questionnaire from at least one of a patient device and a user device, the questions pertaining to a medical condition of a patient;
preparing, by the processor, a medical note interface that includes the received responses, each of the received responses being correlated to a respective question of the plurality of questions;
facilitating, by the processor, provision of the medical note interface to a user;
receiving, by the processor, an indication that the user has reviewed the medical note interface; and
locking, by the processor, the medical note interface responsively to receipt of the indication, the locking preventing modification of the medical note interface.

2. The method of claim 1, further comprising;
receiving, by the processor, a chief complaint of the patient from the at least one patient device and user device; and
adding, by the processor, the chief complaint to the medical note interface.

3. The method of claim 1, wherein the patient is associated with an electronic medical record (EMR), the method further comprising:
associating, by the processor, the medical note interface with the patient's EMR.

4. The method of claim 1, further comprising:
associating, by the processor, the medical note interface with a patient account associated with the patient.

5. The method of claim 1, wherein the responses are received prior to a patient encounter with the user.

6. The method of claim 1, further comprising:
receiving, by the processor, an indication of the time the user spent with the patient during a patient encounter with the user.

7. The method of claim 1, further comprising:
determining, by the processor, a wellness score for the patient based on the received responses.

8. The method of claim 1, further comprising:
receiving, by the processor, narrative comments from the user regarding the patient; and
adding, by the processor, the received comments to the medical note interface.

9. The method of claim 1, further comprising:
accessing, by the processor, a database including rules for determining a level of care delivered to the patient during an encounter with the user;
analyzing, by the processor, the received responses using the accessed rules; and
determining, by the processor, a level of care provided to the patient during the encounter responsively to the analysis.

10. The method of claim 9, further comprising:
facilitating, by the processor, provision of the level of care to at least one of the user and a billing coordinator for the user.

11. The method of claim 9, wherein the rules relate to at least one of the patient's medical history, examination of the patient, medical decision making, time spent with the patient, whether the patient is a new or established patient, patient counseling, user's time spent related to the encounter, and coordination of health care for the patient.

12. The method of claim 9, further comprising:
associating, by the processor, the level of care with the medical note interface.

13. The method of claim 9, further comprising:
determining, by the processor, billing information for the encounter responsively to the determined level of care.

14. The method of claim 1, wherein the medical questionnaire is an outcome measurement device.

15. A method comprising:
receiving, by a processor, a set of responses to a plurality of questions included in a medical questionnaire from at least one of a patient device and a user device, the questions pertaining to a medical condition of a patient;
determining, by the processor, a wellness score for the patient responsively to the set of received responses;
preparing, by the processor, a medical note interface that includes the wellness score for the patient; and
facilitating, by the processor, provision of the medical note interface to a user.

16. The method of claim 15, comprising:
receiving, by the processor, an indication that the user has reviewed the medical note interface; and
locking, by the processor, the medical note interface responsively to receipt of the indication, the locking preventing modification of the medical note interface.

17. The method of claim 15, wherein the set of responses is a first set of responses and the determined wellness score is a first wellness score, the method further comprising:
receiving, by a processor, a second set of responses to the plurality of questions included in the medical questionnaire from at least one of the patient device and the user device;
determining, by the processor, a second wellness score for the patient responsively to the second set of received responses;
determining, by the processor, an improvement score for the patient using the first and second wellness scores; and
adding, by the processor, at least one of the second wellness score and the improvement score to the medical note interface.

18. The method of claim 17, further comprising;
receiving, by the processor, a chief complaint of the patient from the at least one patient device and user device; and
adding, by the processor, the chief complaint to the medical note interface.

19. The method of claim 17, wherein the patient is associated with an electronic medical record (EMR), the method further comprising:
associating, by the processor, the medical note interface with the patient's EMR.

20. The method of claim 17, further comprising:
receiving, by the processor, narrative comments from the user regarding the patient; and
adding, by the processor, the received comments to the medical note interface.
